(19) United States
(12) Patent Application Publication
(10) Pub. No.: US 2021/0338699 A1
(43) Pub. Date: Nov. 4, 2021

(54) MATERIALS AND METHODS FOR SUPPRESSING AND/OR TREATING CANCER AND/OR CANCER TREATMENT INDUCED CARDIAC TOXICITY

(71) Applicant: The Trustees of Indiana University, Indianapolis, IN (US)

(72) Inventors: Reuben Kapur, Zionsville, IN (US); Lei Wei, Fishers, IN (US); Jianjian Shi, Fishers, IN (US)

(73) Assignee: The Trustees of Indiana University, Indianapolis, IN (US)

(21) Appl. No.: 17/271,785

(22) PCT Filed: Aug. 29, 2019
(86) PCT No.: PCT/US2019/048758

§ 371 Date: Feb. 26, 2021

Related U.S. Application Data
(60) Provisional application No. 62/725,222, filed on Aug. 30, 2018.

(51) Int. Cl. A61K 31/704; A61K 31/22; A61K 31/40; A61K 31/44; A61K 31/5025; A61K 31/506; A61K 31/551; A61P 35/00

(52) U.S. Cl. CPC A61K 31/704; A61K 31/22; A61K 31/40; A61P 35/00; A61K 31/5025; A61K 31/506; A61K 31/551; A61K 31/44

(57) ABSTRACT

Various aspects and embodiments disclosed herein relate generally to the modelling, treatment, reducing resistance to the treatment, prevention, and diagnosis of diseases characterized by the formation of cancers and cancer treatment induced symptoms including cardiotoxicity. Embodiments include methods of treating cancer, comprising the steps of: providing a patient diagnosed with cancer with a therapeutic regime that includes at least one therapeutically effective dose of at least one agent that reduces the activity of at least one Rho/Rho kinase pathway. Other embodiments include methods of treating cancer, comprising the steps of: treating a patient diagnosed with cancer with a combination of therapeutic agents that includes at least one therapeutically effective anti-cancer agent and at least one compound that reduces the activity of at least one Rho/Rho kinase pathway. Yet other embodiments include methods of reducing cancer treatment induced cardiotoxicity, comprising the steps of: treating a patient at least one therapeutically effective dose of at least one agent that reduces the activity of at least one Rho/Rho kinase pathway.

### CROSS-REFERENCE TO RELATED APPLICATIONS

This application claims the benefit of U.S. Provisional Application No. 62/725,222, filed October Aug. 30, 2018, the entire disclosure of which is hereby expressly incorporated by reference herein.

### FIELD OF THE INVENTION

Various aspects and embodiments disclosed herein relate generally to the modelling, treatment, reducing resistance to the treatment, prevention, and diagnosis of diseases characterized by the formation of cancers and cancer treatment induced symptoms including cardiotoxicity.

### BACKGROUND AND SUMMARY

Anthracyclines (ANTs) such as doxorubicin (DOX) and epirubicin are among the most effective anticancer treatments and are effective against many types of cancer. ANTs are considered front line therapy for the treatment of a variety of hematologic malignancies including acute myeloid leukemia (AML). Unfortunately, the clinical use of ANTs is limited by cumulative, dose-related cardiotoxicity (CTX), which often results in severe and irreversible form of cardiomyopathy. Children are particularly susceptible to the CTX effects of DOX, and no safe dose of ANTs has been reported for this population. Young children under the age of four appear to be especially vulnerable as well as children with Down syndrome (DS). About half of young adult survivors of childhood cancer have received ANTs at some point during their treatment, and long-term follow up studies show a progressive development of CTX in majority of children treated with ANTs.

In addition, targeted tyrosine kinase inhibitor (TKI) therapies such as those approved for treating Philadelphia positive (Ph+) chronic myelogenous leukemia (CML) and Ph+ acute lymphocytic leukemia (ALL) (e.g., Imatinib, Dasatinib, Nilotinib, and Ponatinib) have revolutionized the treatment of these diseases. Unfortunately, a long-term exposure of Imatinib (1st generation TKI) and Dasatinib (2nd generation TKI) in CML and ALL patients has led to the development of drug resistance, mostly due to acquisition of new mutations. To circumvent drug resistance associated with the first and the second generation TKIs, a third generation TKI, Ponatinib, was developed. Although Ponatinib is highly effective for treating refractory CML and ALL, treatment with Ponatinib resulted in severe CTX including vascular injury. Accordingly, both non-specific therapies (e.g., DOX) and targeted therapies (e.g., second and third generation TKIs) have resulted in CTX during the treatments. Despite intensive investigations on ANT- and/or TKI-induced CTX, the underlying mechanisms remain poorly understood. Thus, new strategies providing effective prevention of cardiac side effects continue to be in great demand.

A first embodiment includes a composition, comprising at least one anti-cancer agent; and at least one agent that reduces the activity of at least one Rho/Rho Kinase pathway.

A second embodiment includes the composition according to the first embodiment, wherein the at least one anti-cancer agent comprises at least one anthracycline (ANT) and/or at least one tyrosine kinase inhibitor (TKI).

A third embodiment includes the composition according to any one of the first and the second embodiments, wherein the at least one anti-cancer agent includes at least one anthracycline (ANT), and wherein the at least one anthracycline (ANT) includes, but is not limited to, daunorubicin, doxorubicin, epirubicin, idarubicin, pixantrone, sabarubicin, and valrubicin, or a derivative thereof, or any combination thereof.

A fourth embodiment includes the composition according to any one of the first to the third embodiments, wherein the at least one anti-cancer agent includes at least one tyrosine kinase inhibitor (TKI), and wherein the at least one tyrosine kinase inhibitor (TKI) includes, but is not limited to, imatinib, dasatinib, nilotinib, bosutinib, ponatinib, lapatinib, adavosertib, gefitinib, erlotinib, toceranib, sorafenib, nilotinib, afatinib, axitinib, cabozantinib, osimertinib, lenvatinib, midostaurin, neratinib, regorafenib, and vandetanib, or a derivative thereof, or any combination thereof.

A fifth embodiment includes the composition according to any one of the first to the fourth embodiments, wherein the at least one agent that reduces the activity of the Rho/Rho kinase pathway comprises at least one Rho inhibitor and/or at least one Rho kinase (ROCK) inhibitor.

A sixth embodiment includes the composition according to any one of the first to the fifth embodiments, wherein the at least one agent that reduces the activity of at least one Rho/Rho Kinase pathway includes at least one Rho inhibitor, and wherein the at least one Rho inhibitor includes, but is not limited to, rhosin, Y16, and C3 Transferases, or a derivative thereof, or any combination thereof.

A seventh embodiment includes the composition according to any one of the first to the sixth embodiments, wherein the at least one agent that reduces the activity of at least one Rho/Rho Kinase pathway includes at least one Rho kinase (ROCK) inhibitor, and wherein the at least one Rho kinase (ROCK) inhibitor includes, but is not limited to, Fasudil, H-1152, Ripasudil, RKI-1447, Y-27632, GSK429286A, Y-30141, and nitric oxide (NO), or a derivative thereof, or any combination thereof.

An eighth embodiment includes the composition according to any one of the first to the seventh embodiments, further comprising at least one statin. Consistent with these embodiments, the at least one statin includes, but is not limited to, atorvastatin, fluvastatin, lovastatin, pitavastatin, pravastatin, rosuvastatin, simvastatin, and ezetimibe/simvastatin combination, or a derivative thereof, or any combination thereof.

A ninth embodiment includes the composition according to any one of the first to the eighth embodiments, wherein the at least one anti-cancer agent and the at least one agent that reduces the activity of at least one Rho/Rho Kinase pathway are present in the composition in a ratio such that the composition exhibits synergy. The ratio of the at least one anti-cancer agent and the at least one agent that reduces the activity of at least one Rho/Rho Kinase pathway can be from about 10:1 to about 1:10, from about 9:1 to about 1:9, from about 8:1 to 1:8, from about 5:1 to about 1:5, from about 2:1 to about 1:2, about 10:1, 5:1, 2:1, 1:1, 1:2, 1:5, 1:10, or any combination(s) thereof.

A tenth embodiment includes a method of treating a cancer, comprising the steps of: administering to a subject diagnosed with a cancer a therapeutically effective dose of the composition according to any one of the first to the ninth embodiments or a pharmaceutically acceptable salt or metabolite thereof.

An eleventh embodiment includes the method according to the tenth embodiment, wherein the subject comprises a human, an animal, a livestock, an organ, a cell, and/or a tissue.

A twelfth embodiment includes the method according to any one of the tenth and the eleventh embodiments, wherein the cancer includes, but is not limited to, bone cancer, brain cancer, breast cancer, endocrine cancer, gastrointestinal cancer, gynaecologic cancer, head and neck cancer, hematologic cancer, lung cancer, prostate cancer, renal cell carcinoma, skin cancer, urologic cancer, rare cancers, hematologic malignancies, lymphoma, leukemia, acute lymphoblastic leukemia (ALL), acute myeloid leukemia (AML), chronic lymphocytic leukemia (CLL), and chronic myeloid leukemia (CML), or any combination thereof.

A thirteenth embodiment includes the method according to any one of the tenth to the twelfth embodiments, wherein the therapeutically effective dose of the composition according to any one of the first to the ninth embodiments includes from 10 mg/kg/day to 1000 mg/kg/day, from 10 mg/kg/day to 500 mg/kg/day, from 10 mg/kg/day to 200 mg/kg/day, from 10 mg/kg/day to 100 mg/kg/day, from 10 mg/kg/day to 50 mg/kg/day, from 20 mg/kg/day to 1000 mg/kg/day, from 20 mg/kg/day to 500 mg/kg/day, from 20 mg/kg/day to 200 mg/kg/day, from 20 mg/kg/day to 10 mg/kg/day, 20 mg/kg/day, 30 mg/kg/day, 40 mg/kg/day, 50 mg/kg/day, 60 mg/kg/day, 70 mg/kg/day, 80 mg/kg/day, 90 mg/kg/day, 100 mg/kg/day, 110 mg/kg/day, 120 mg/kg/day, 130 mg/kg/day, 140 mg/kg/day, 150 mg/kg/day, 200 mg/kg/day, 300 mg/kg/day, 400 mg/kg/day, 500 mg/kg/day, 600 mg/kg/day, 700 mg/kg/day, 800 mg/kg/day, 900 mg/kg/day, 1000 mg/kg/day, or any combination thereof. In these embodiments, the effective dose can be on the order of between about 5 mg to about 2000 mg and the dose of the composition according to any one of the first to the ninth embodiments is administered to the patient at least once, twice, or three times per day. In some embodiments, the therapeutically effective dose of the composition according to any one of the first to the ninth embodiments, includes, but is not limited to, on the order of between: about 10 mg to about 1900 mg; about 15 mg to about 1800 mg; about 15 mg to about 1700 mg; about 20 mg to about 1600 mg; about 25 mg to about 1500 mg; about 30 mg to about 1000 mg; about 50 mg to about 1000 mg; about 50 mg to about 800 mg; about 100 mg to about 800 mg; about 300 mg to about 800 mg, about 500 mg to about 800 mg; about 5 mg to about 50 mg; about 1000 mg to about 1700 mg; about 1200 mg to about 1700 mg; about 1500 mg to about 1700 mg; about 10 mg to about 1000 mg; about 10 mg to about 30 mg; about 1500 mg to about 2000 mg; about 100 mg to about 200 mg; about 100 mg to about 150 mg; and/or any combination thereof. Consistent with these embodiments, the therapeutically effective dose of the composition according to any one of the first to the ninth embodiments, includes, but not limited to, on the order of between: about 1 mg/m² to about 1500 mg/m²; about 10 mg/m² to about 1000 mg/m²; about 20 mg/m² to about 800 mg/m²; about 10 mg/m² to about 50 mg/m²; about 800 mg/m² to about 1200 mg/m²; about 50 mg/m² to about 500 mg/m²; about 500 mg/m² to about 1000 mg/m²; about 80 mg/m² to about 150 mg/m²; about 80 mg/m² to about 120 mg/m²; and/or any combination thereof.

A fourteenth embodiment includes the method according to any one of the tenth to the thirteenth embodiments, wherein the composition of any one of the first to the ninth embodiments is administered orally, parenterally, rectally, and/or transdermally.

A fifteenth embodiment includes a method of reducing a side effect of a therapeutic regime, comprising the steps of: administering to a subject at least one therapeutically effective dose of at least one agent that reduces the activity of at least one Rho/Rho kinase pathway; wherein the subject has received at least one therapeutic regime comprising surgery, chemotherapy, radiation therapy, bone marrow transplant, targeted therapy, precision medicine, immunotherapy, stem cell transplant, hyperthermia, photodynamic therapy, blood product donation and transfusion, Light Amplification by Stimulated Emission of Radiation (LASER) in cancer treatment, DNA-damaging radiotherapy, endocrine therapy, and/or hormone therapy, and wherein the subject experiences at least one side effect.

A sixteenth embodiment includes the method according to the fifteenth embodiment, wherein the at least one therapeutically effective dose of at least one agent that reduces the activity of at least one Rho/Rho Kinase pathway includes, but is not limited to, at least one Rho inhibitor and at least one Rho kinase (ROCK) inhibitor.

A seventeenth embodiment includes the method according to any one of the fifteenth and the sixteenth embodiments, wherein the at least one agent that reduces the activity of at least one Rho/Rho Kinase pathway includes at least one Rho inhibitor, and wherein the at least one Rho inhibitor includes, but is not limited to, rhosin, Y16, and/or C3 Transferases, or a derivative and/or a pharmaceutical salt thereof, or any combination thereof.

An eighteenth embodiment includes the method according to any one of the fifteenth embodiment to the seventeenth embodiments, wherein the at least one agent that reduces the activity of at least one Rho/Rho Kinase pathway includes at least one Rho kinase (ROCK) inhibitor, and wherein the at least one Rho kinase (ROCK) inhibitor includes, but is not limited to, Fasudil, H-1152, Ripasudil, RKI-1447, Y-27632, GSK429286A, Y-30141, and/or nitric oxide (NO), or a derivative and/or a pharmaceutical salt thereof, or any combination thereof.

A nineteenth embodiment includes the method according to any one of the fifteenth to the eighteenth embodiments, wherein the therapeutic regime comprises administration of at least one therapeutic agent comprising at least one anthracycline (ANT) and/or at least one tyrosine kinase inhibitor (TKI). Consistent with these embodiments, the administration of at least one therapeutic agent comprising at least one anthracycline (ANT) and/or at least one tyrosine kinase inhibitor (TKI) induces the side effect.

A twentieth embodiment includes the method according to any one of the fifteenth to the nineteenth embodiments, wherein the at least one anthracycline (ANT) includes, but is not limited to, daunorubicin, doxorubicin, epirubicin, idarubicin, pixantrone, sabarubicin, and/or valrubicin, or a derivative thereof, or any combination thereof.

A twenty first embodiment includes the method according to any one of the fifteenth to the twentieth embodiments, wherein the at least one tyrosine kinase inhibitor (TKI) includes, but is not limited to, imatinib, dasatinib, nilotinib, bosutinib, ponatinib, lapatinib, adavosertib, gefitinib, erlotinib, toceranib, sorafenib, nilotinib, afatinib, axitinib, cabozantinib, osimertinib, lenvatinib, midostaurin, neratinib, regorafenib, and/or vandetanib, or a derivative thereof, or any combination thereof.

A twenty second embodiment includes the method according to any one of the fifteenth to the twenty first embodiments, wherein the therapeutically effective dose of the at least one agent that reduces the activity of at least one Rho/Rho Kinase pathway includes from 10 mg/kg/day to 1000 mg/kg/day, from 10 mg/kg/day to 500 mg/kg/day, from 10 mg/kg/day to 200 mg/kg/day, from 10 mg/kg/day to 100 mg/kg/day, from 10 mg/kg/day to 50 mg/kg/day, from 20 mg/kg/day to 1000 mg/kg/day, from 20 mg/kg/day to 500 mg/kg/day, from 20 mg/kg/day to 200 mg/kg/day, from 20 mg/kg/day to 10 mg/kg/day, 20 mg/kg/day, 30 mg/kg/day, 40 mg/kg/day, 50 mg/kg/day, 60 mg/kg/day, 70 mg/kg/day, 80 mg/kg/day, 90 mg/kg/day, 100 mg/kg/day, 110 mg/kg/day, 120 mg/kg/day, 130 mg/kg/day, 140 mg/kg/day, 150 mg/kg/day, 200 mg/kg/day, 300 mg/kg/day, 400 mg/kg/day, 500 mg/kg/day, 600 mg/kg/day, 700 mg/kg/day, 800 mg/kg/day, 900 mg/kg/day, 1000 mg/kg/day, or any combination thereof. In these embodiments, the effective dose can be on the order of between about 5 mg to about 2000 mg and the dose of the at least one agent that reduces the activity of at least one Rho/Rho Kinase pathway is administered to the patient at least once, twice, or three times per day. In some embodiments, the therapeutically effective dose of the at least one agent that reduces the activity of at least one Rho/Rho Kinase pathway, includes, but is not limited to, on the order of between: about 10 mg to about 1900 mg; about 15 mg to about 1800 mg; about 15 mg to about 1700 mg; about 20 mg to about 1600 mg; about 25 mg to about 1500 mg; about 30 mg to about 1000 mg; about 50 mg to about 1000 mg; about 50 mg to about 800 mg; about 100 mg to about 800 mg; about 300 mg to about 800 mg; about 500 mg to about 800 mg; about 5 mg to about 50 mg; about 1000 mg to about 1700 mg; about 1200 mg to about 1700 mg; about 1500 mg to about 1700 mg; about 10 mg to about 1000 mg; about 10 mg to about 30 mg; about 1500 mg to about 2000 mg; about 100 mg to about 200 mg; about 100 mg to about 150 mg; and/or any combination thereof. Consistent with these embodiments, the therapeutically effective dose of the at least one agent that reduces the activity of at least one Rho/Rho Kinase pathway, includes, but not limited to, on the order of between: about 1 mg/m² to about 1500 mg/m²; about 10 mg/m² to about 1000 mg/m²; about 20 mg/m² to about 800 mg/m²; about 10 mg/m² to about 50 mg/m²; about 800 mg/m² to about 1200 mg/m²; about 50 mg/m² to about 500 mg/m²; about 500 mg/m² to about 1000 mg/m²; about 80 mg/m² to about 150 mg/m²; about 80 mg/m² to about 120 mg/m²; and/or any combination thereof.

A twenty third embodiment includes the method according to any one of fifteenth to the twenty second embodiments, wherein the at least one side effect includes, but not limited to, drug-resistance, relapse, cardiotoxicity, and/or cardiomyopathy.

A twenty fourth embodiment includes the method according to any one of the first to the twenty third embodiments, wherein the subject comprises a human, an animal, a livestock, an adult, or a child.

### BRIEF DESCRIPTION OF THE FIGURES

FIG. 1A. Schematic diagram representing serial doxorubicin administration protocol. WT and ROCK1^{-/-} mice received three serial injections weekly of normal saline (NS) or doxorubicin (DOX) (8 mg/kg). Mice were sacrificed on day 21 after the initial injection.

FIG. 1B. Representative short-zxis echocardiograms illustrating the effect of DOX before each injection (WT-Baseline), 7 days after the initial injection (WT-DOX day 7), 14 days after the initial injection (WT-DOX day 14), 21 days after the initial injection (WT-DOX day 21).

FIG. 1C. Graph illustrating the effect on percent fractional shortening (FS) at indicated time points. Cardiac function in WT mice, but not in ROCK1^{-/-} mice, was impaired on day 14 (i.e., 1 week after the 2^{nd} dose). N=10-15 in each group. *p<0.05 for DOX-injected vs. NS-injected mice. #p<0.05 for DOX-ROCK1^{-/-} mice vs. DOX-WT mice.

FIG. 1D. Graph illustrating the effect on body weight at indicated time points. DOX affected body weight on day 21 after the initial injection in both WT and ROCK1^{-/-} mice. N=10-15 in each group. *p<0.05 for DOX-injected vs. NS-injected mice. #p<0.05 for DOX-ROCK1^{-/-} mice vs. DOX-WT mice.

FIG. 2A. Graph illustrating the effect on left ventricular end systolic dimension (LVESD) of WT and ROCK1^{-/-} mice on day 21 after the initial injection. Cardiac dimension was preserved in COX-treated ROCK1^{-/-} mice compared to WT mice. N=8-10 in each group.

FIG. 2B. Graph illustrating the effect on left ventricular end diastolic dimension (LVEDD) of WT and ROCK1^{-/-} mice on day 21 after the initial injection. Cardiac dimension was preserved in COX-treated ROCK1^{-/-} mice compared to WT mice. N=8-10 in each group.

FIG. 2C. Graph illustrating quantitative analysis of heart weight of WT and ROCK1^{-/-} mice on day 21 after the initial injection. N=10 in each group. p<0.01 for DOX-injected ROCK1^{-/-} mice vs. DOX-injected WT mice.

FIG. 2D. Graph illustrating quantitative analysis of heart weight per tibial length (TL) ratio (Heart wt/TL) of WT and ROCK1^{-/-} mice on day 21 after the initial injection. N=10 in each group. p<0.01 for DOX-injected ROCK1^{-/-} mice vs. DOX-injected WT mice.

FIG. 3A. Graph illustrating quantitative analysis of cardiac myocyte area measured from laminin stained sections. Each column represents results obtained from approximately 200 myocytes from at least four hearts per group. ROCK1 deficiency does not prevent DOX-induced reduction in heart weight and cardiomyocyte size.

FIG. 3B. Graph illustrating percent survival indicated days after the treatment. Kaplan-Meier survival curves (over 6 weeks after the initial DOX injection) indicate that the mortality rate was significantly lower in the ROCK1^{-/-} mice than that in WT mice. N=10 in each group. p<0.01 for DOX-injected ROCK1^{-/-} mice vs. DOX-injected WT mice.

FIG. 4A. Graph illustrating quantification of total TUNEL positive nuclei per 10⁵ total nuclei in ventricular myocardium from WT and ROCK1^{-/-} hearts on day 21 after the initial injection. N=4-6 in each group.

FIG. 4B. Western blot (top) illustrating the effect on Bax and Cox IV in mitochondrial fraction of ventricular homogenates from WT and ROCK1−/− hearts. Quantitative analysis (bottom) of immune-reactive bands of Bax (N=4-6 in each group) expressed as fold change relative to NS-treated WT group.

FIG. 4C. Representative heart sections (top) stained with picrosirius red/Fast green (scale bar, 50 μm) showing collagen deposition. Quantitative analysis (bottom) of the collagen deposition in WT and ROCK1^{-/-} hearts, expressed as percent change relative to NS-treated WT hearts. N=4-6 hearts in each group. At least 10 randomly chosen high power fields per section and four transverse sections from each heart, sampled from the midpoint between the apex and base, were analyzed.

FIG. 5A. Western blot illustrating the effect on ROCK1, ROCK2, LC3, Beclin 1, p-AMPK-Thr172, Bax, FAK, and p-FAK-Tyr397 in ventricular homogenates of WT and ROCK1^{-/-} hearts on day 21 after the initial injection.

FIG. 5B. Graph illustrating quantitative analysis of ROCK1.

FIG. 5C. Graph illustrating quantitative analysis of Bax.

FIG. 5D. Graph illustrating quantitative analysis of p-FAK-Tyr397/FAK.

FIG. 5E. Graph illustrating quantitative analysis of LC3-II.

FIG. 5F. Representative transmission electron microscopy images illustrating the effect on WT and ROCK1^{-/-} hearts on day 21 after the initial injection. DOX-treated WT heart showed increased numbers of autophagic vacuoles (arrows). Scale bar, 0.5 μm.

FIG. 6A. Southern blot illustrating the effect on genomic DNA obtained from the tail of WT and ROCK1^{fl-neo/+} mice.

FIG. 6B. Western blot illustrating the ROCK1 levels in the heart and tail of WT, ROCK1^{fl/fl}, and MHC-Cre/ROCK1^{fl/fl} mice. This shows about 80% reduction of ROCK1 expression in the heart samples of MHC-Cre/ROCK1^{fl/fl} mice compared with WT or ROCK1^{fl/fl} mice, but not in the tail samples of these mice. Residual ROCK1 expression in the heart can be due in part to the presence of ROCK1 in other cell types in hearts (e.g., fibroblasts, vascular endothelial cells, and inflammatory cells).

FIG. 7A. Graph illustrating the percent fractional shortening (FS) of cardiomyocyte-specific ROCK1 knockout mice (MHC-Cre/ROCKfl/fl) and ROCK1fl/fl mice (8-9 weeks old) after receiving three serial injections weekly of NS or DOX (8 mg/kg). Cardiac function was measured by echocardiography analysis on day 21 after the initial injection.

FIG. 7B. Graph illustrating total TUNEL positive nuclei per 10⁵ total nuclei in ventricular myocardium from MHC-Cre/ROCK^{fl/fl} and ROCK1^{fl/fl} mice hearts on day 21. N=4-6 in each group.

FIG. 7C. Representative heart sections stained with picrosirius red/Fast green illustrating the collagen deposition in ventricular myocardium of MHC-Cre/ROCK^{fl/fl} and ROCK1^{fl/fl} mice hearts on day 21. N=4-6 in each group.

FIG. 8A. Western blot illustrating the effect of H-1152 on phosphorylation of MYPT1 in myeloid cells bearing empty vector, FLT3, BCR-Able or FLT3N51. Myeloid cells bearing empty vector, FLT3, BCR-Able or FLT3N51 were starved for 6 hours in serum- and cytokine-free medium and incubated in the presence or absence of H-1152 (2 μM) for 1 hour and equal amount of protein lysates were subjected to Western blot analysis. N=3.

FIG. 8B. Western blot illustrating the effect of H-1152 on phosphorylation of AKT and Stat5 in myeloid cells bearing empty vector, FLT3, BCR-Able or FLT3N51. Myeloid cells bearing empty vector, FLT3, BCR-Able or FLT3N51 were starved for 6 hours in serum- and cytokine-free medium and incubated in the presence or absence of H-1152 (2 μM) for 1 hour and equal amount of protein lystates were subjected to Western blot analysis. N=3.

FIG. 8C. Western blot illustrating the effect of H-1152 on phosphorylation of PKC in myeloid cells bearing empty vector, FLT3, BCR-Able or FLT3N51. Myeloid cells bearing empty vector, FLT3, BCR-Able or FLT3N51 were starved for 6 hours in serum- and cytokine-free medium and incubated in the presence or absence of H-1152 (2 μM) for 1 hour and equal amount of protein lysates were subjected to Western blot analysis. N=3.

FIG. 9A. Graphs illustrating the effect of H-1152 on proliferation of myeloid cells bearing empty vector, FLT3, BCR-Able or FLT3N51. After 48 hours, proliferation was evaluated by [³H] thymidine incorporation.

FIG. 9B. Graph illustrating the growth comparison of 32D cells bearing BCR-ABLT315I in the presence of indicated concentrations of Imatinib (IM) and H-1152. Bars denote the mean thymidine incorporation f SD from 1 of 3 independent experiments in quadruplicate. *p<0.01.

FIG. 10A. Graph illustrating the effect of H-1152 on proliferation of MV4-11 cells. After 48 hours, proliferation was evaluated by [³H] thymidine incorporation. Bars denote the mean thymidine incorporation f SD from a representative experiment performed in quadruplicate. N=3, *p<0.001.

FIG. 10B. Graphs illustrating three representative AML patient samples positive for FLT3ITD mutation that were grown in the presence of indicated cytokines and treated with indicated amounts of H-1152. After 48 hours, proliferation was evaluated. Bars denote the mean thymidine incorporation f SD performed in triplicate or quadruplicate. *p<0.01.

FIG. 11A. Graph illustrating the effect of H-1152 on proliferation of K562 cells. After 48 hours, proliferation was evaluated by [³H] thymidine incorporation. Bars denote the mean thymidine incorporation f SD from a representative experiment performed in quadruplicate. N=2, *p<0.001.

FIG. 11B. Graph illustrating the effect of H-1152 on proliferation of CML patient samples positive for BCR-ABL. After 48 hours, proliferation was evaluated by [³H] thymidine incorporation. Bars denote the mean thymidine incorporation±SD performed in triplicate or quadruplicate. *p<0.01.

FIG. 11C. Graph illustrating the effect of H-1152 and cytokines on proliferation of CML patient samples positive for BCR-ABL. After 48 hours, proliferation was evaluated by [³H] thymidine incorporation. Bars denote the mean thymidine incorporation f SD performed in triplicate or quadruplicate. *p<0.01.

FIG. 12A. Graph illustrating the effect of ROCK inhibitor (H-1152) alone or in combination with imatinib on proliferation of myeloid cells expressing BCR-ABL.

FIG. 12B. Graph illustrating the effect of ROCK inhibitor (H-1152) alone or in combination with imatinib on proliferation of myeloid cells expressing BCR-ABL T315I.

FIG. 12C. Graph illustrating the effect of ROCK inhibitor (H-1152) on percent survival of mice bearing cells expressing BCR-ABL T315I. In vivo evidence to suggest that pre-treating BCR-ABL T315I mutant expressing cells with the ROCK inhibitor prior to transplantation significantly enhances the survival of these mice.

FIG. 13A. Graph illustrating the effect of ROCK inhibitor (H-1152) on apoptosis of bone marrow (BM) cells bearing vector or BCR-ABL. BM cells bearing vector or BCR-Able were starved for 6 hours in serum- and cytokine-free medium, and treated with indicated amounts of ROCK inhibitor and grown for 48 hours. After 48 hours, apoptosis was analyzed by staining the cells with Annexin V and 7-AAD. Assays were performed in the presence of IL-3 (10 ng/mL) for vector bearing cells and in the absence of IL-3 for BCR-ABL bearing cells. Shown is a representative experiment from one of 3 independent experiments in triplicate. Mean±SD. *p<0.05.

FIG. 13B. Quantitative analysis of cells bearing FLT3 or FLT3N51 by flow cytometry. Cells bearing FLT3 or FLT3N51 were starved for 6 hours in serum- and cytokine-free medium, and treated with indicated amounts of ROCK inhibitor and grown for 48 hours. After 48 hours, apoptosis was analyzed by staining the cells with Annexin V and 7-AAD.

FIG. 14A. Graph illustrating percent survival of WT, Tet2^{-/-}, FLT3^{ITD/ITD} and Tet2^{-/-}FLT3^{ITD/ITD} mice. WT, Tet2^{-/-}, FLT3^{ITD/IDT} and Tet2^{-/-}FLT3^{ITD/ITD} mice were monitored for survival, demonstrated by Kaplan Meir survival curve (n=7-10 mice/group, **p<0.01 comparing Tet2^{-/-}FLT3^{ITD/ITD} to all other genotypes).

FIG. 14B. Graph illustrating bone marrow Light-density mononuclear cells (BM LDMNCs) in WT, Tet2^{-/-}, FLT3^{ITD/ITD} and Tet2^{-/-}FLT3^{ITD/ITD} mice. 4-month-old WT (white), Tet2^{-/-} (blue), FLT3^{ITD/ITD} (green) and Tet2^{-/-}FLT3^{ITD/ITD} (red) mice were analyzed for BM cellularity, BM frequency and absolute number of Lin⁻Sca1⁺ KIT⁺ (LSK) cells, and BM frequency of Lin-Sca1⁺KIT⁺CD48⁻CD150⁺ cells (n=3-6 mice/group±SEM, *p<0.05 compared to TW, **p<0.01 compared to WT).

FIG. 14C. Graph illustrating percent LSK cells in WT, Tet2^{-/-}, FLT3^{ITD/ITD} and Tet2^{-/-}FLT3^{ITD/ITD} mice. 4-month-old WT (white), Tet2^{-/-} (blue), FLT3^{ITD/ITD} (green) and Tet2^{-/-}FLT3^{ITD/ITD} (red) mice were analyzed for BM cellularity, BM frequency and absolute number of Lin-Sca1⁺KIT⁺ (LSK) cells, and BM frequency of Lin-Sca1⁺KIT⁺CD48⁻CD150⁺ cells (n=3-6 mice/group±SEM, *p<0.05 compared to TW, **p<0.01 compared to WT).

FIG. 14D. Graph illustrating number of LSK cells in WT, Tet2^{-/-}, FLT3^{ITD/ITD} and Tet2^{-/-}FLT3^{ITD/ITD} mice. 4-month-old WT (white), Tet2^{-/-} (blue), FLT3^{ITD/ITD} (green) and Tet2^{-/-}FLT3^{ITD/ITD} (red) mice were analyzed for BM cellularity, BM frequency and absolute number of Lin⁻Sca1⁺KIT⁺ (LSK) cells, and BM frequency of Lin⁻Sca1⁺KIT⁺CD48⁻CD150⁺ cells (n=3-6 mice/group±SEM, *p<0.05 compared to TW, **p<0.01 compared to WT).

FIG. 14E. Graph illustrating percent Lin⁻Sca1⁺Kit⁺CD48⁻CD150⁺ cells in WT, Tet2^{-/-}, FLT3^{ITD/ITD} and Tet2^{-/-}FLT3^{ITD/ITD} mice. 4-month-old WT (white), Tet2^{-/-} (blue), FLT3^{ITD/ITD} (green) and Tet2^{-/-}FLT3^{ITD/ITD} (red) mice were analyzed for BM cellularity, BM frequency and absolute number of Lin⁻Sca1⁺KIT⁺ (LSK) cells, and BM frequency of Lin⁻Sca1⁺KIT⁺CD48⁻CD150⁺ cells (n=3-6 mice/group±SEM, *p<0.05 compared to TW, **p<0.01 compared to WT).

FIG. 14F. Quantitative analysis of KIT⁺Mac1⁺ immature myeloid blasts of 4-month-old WT (white), Tet2^{-/-} (blue), FLT3^{ITD/ITD} (green) and Tet2^{-/-}FLT3^{ITD/ITD} (red) mice by flow cytometry. Spleen from 4-month-old WT (white), Tet2^{-/-} (blue), FLT3^{ITD/ITD} (green) and Tet2^{-/-}FLT3^{ITD/ITD} (red) mice were analyzed for KIT⁺Mac1⁺ immature myeloid blasts (n=3 mice/group, **p<0.01 compared to WT).

FIG. 14G. Graph illustrating percent survival of mice transplanted with bone marrow Light-density mononuclear cells (BM LDMNCs) of WT, Tet2^{-/-}, FLT3^{ITD/ITD} and Tet2^{-/-}FLT3^{ITD/ITD} mice. BM LDMNCs from WT, Tet2^{-/-}, FLT3^{ITD/ITD} and Tet2^{-/-}FLT3^{ITD/ITD} mice were transplanted into lethally irradiated C57BL/6 mice and monitored for disease progression and survival. Shown is Kaplan Meier survival curve (n=5 mice/group, **p<0.05 comparing Tet2^{+/-}:FLT3^{ITD/WT} to all other genotypes).

FIG. 15A. Graph illustrating percent survival of mice transplanted with bone marrow Light-density mononuclear cells (BM LDMNCs) from polyI:polyC-treated WT, Dnmt3a^{+/-}, FLT3^{ITD/+} and DNMT3a^{+/-}FLT3^{ITD/+} mice. BM LDMNCs (2×10⁶) from polyI:polyC-treated (4 month) WT, Dnmt3a^{+/-}, FLT3^{ITD/+} and DNMT3a^{+/-}FLT3^{ITD/+} mice were transplanted into lethally irradiated C57BL/6 mice and monitored for disease progression and survival. Shown is Kaplan Meier survival curve (n=3-4 mice/group, *p<0.05 comparing DNMT3a^{+/-} FLT3^{ITD/+} to all other genotypes).

FIG. 15B. Graph illustrating the effect of polyI:polyC treatment on spleen weight. WT (white), Dnmt3a^{+/-} (black), FLT3^{ITD/+} (blue) and DNMT3a^{+/-}FLT3^{ITD/+} (red) mice were polyI:polyC-treated and analyzed after 4 months for splenomegaly (n=6-7 mice/group±SEM, **p<0.05 compared WT).

FIG. 15C. Graph illustrating the effect of polyI:polyC treatment on bone marrow LDMNCs. WT (white), Dnmt3a^{+/-} (black), FLT3^{ITD/+} (blue) and DNMT3a^{+/-}FLT3^{ITD/+} (red) mice were polyI:polyC-treated and analyzed after 4 months for BM cellularity (n=6-7 mice/group±SEM, **p<0.05 compared WT).

FIG. 15D. Quantitative analysis of KIT⁺Mac1⁺ immature myeloid blasts of WT (white), Dnmt3a^{+/-} (black), FLT3^{ITD/+} (blue) and DNMT3a^{+/-}FLT3^{ITD/+} (red) mice. WT (white), Dnmt3a^{+/-} (black), FLT3^{ITD/+} (blue) and DNMT3a^{+/-}FLT3^{ITD/+} (red) mice were polyI:polyC-treated and analyzed after 4 months (n=6-7 mice/group±SEM, **p<0.05 compared WT).

FIG. 16. Electrophoresis gel validating MHC-Cre/ROCK^{fl/fl} and Tie2-Cre/ROCK^{fl/fl} in the hearts of 16-week old mice. ROCK1 expression in hearts showed about 50% reduction and about 30% reduction, respectively. p<0.05 vs. ROCK1^{fl/fl}. Remaining residual ROCK1 expression is due to presence of other ROCK1 expressing cells in the heart.

FIG. 17. Electrophoresis gel validating MHC-ROCK1 transgenic mice with about 5 fold increase of ROCK1 in heart, associated with increased ROCK activity as measured by p-MLC levels. p<0.05 vs. No Treatment Group (NTG).

FIG. 18. Schematic drawing illustrating ABL-dependent regulation of the Rho-ROCK-myosin signalling pathway.

FIG. 19A. Immunostaining illustrating the effect of ROCK1 deficiency. ROCK1 deficiency reduced Ponatinib-induced cell shape changes. WT cells, but not ROCK1 deficient cells, exhibited increased formation of cortical contractile rings and reduced cell size.

FIG. 19B. Quantification of electrophoresis gel illustrating the effect of ROCK1 deficiency. Ponatinib induced an increase in p-MYPT1 level in WT cells but not in ROCK1 deficient cells. ROCK1 deficient cells were more resistant to Ponatinib-induced activation of caspases 3 and 8. *p<0.05.

FIG. 20. Graphs illustrating percent survival of leukemic mice treated with Fasudil or H-1152 or leukemic cells lacking ROCK1 compared to vehicle treated WT leukemic cells.

### DESCRIPTION

For the purposes of promoting an understanding of the principles of the novel technology, reference will now be made to the preferred embodiments thereof, and specific language will be used to describe the same. It will nevertheless be understood that no limitation of the scope of the novel technology is thereby intended, such alterations, modifications, and further applications of the principles of the novel technology being contemplated as would normally occur to one skilled in the art to which the novel technology relates are within the scope of this disclosure and the claims.

As used herein, unless explicitly stated otherwise or clearly implied otherwise the term ‘about’ refers to a range of values plus or minus 10 percent, e.g. about 1.0 encompasses values from 0.9 to 1.1.

The term, “treating” as used herein unless stated or implied otherwise, includes administering to a human or an animal patient at least one dose of a compound, treating includes preventing or lessening the likelihood and/or severity of at least one disease as well as limiting the length of an illness or the severity of an illness, treating may or may not result in a cure of the disease.

As used herein, unless explicitly stated otherwise or clearly implied otherwise the terms ‘therapeutically effective dose,’ ‘therapeutically effective amounts,’ and the like, refer to a portion of a compound that has a net positive effect on health and well being of a human or other animal. Therapeutic effects may include an improvement in longevity, quality of life and the like these effects also may also include a reduced susceptibility to developing disease or deteriorating health or well being. The effects may be immediate realized after a single dose and/or treatment or they may be cumulative realized after a series of doses and/or treatments. A “therapeutically effective amount” in general means the amount that, when administered to a subject or animal for treating a disease, is sufficient to affect the desired degree of treatment for the disease.

As used herein, “inhibition” or “inhibitory activity” each encompass whole or partial reduction of activity or effect of an enzyme or all and/or part of a pathway that includes an enzyme that is effected either directly or indirectly by the inhibitor or a pathway that is effected either directly or indirectly by the activity of the enzyme which is effected either directly or indirectly by the inhibitor.

As used herein, the term “pharmaceutically acceptable salt” is defined as a salt wherein the desired biological activity of the inhibitor is maintained and which exhibits a minimum of undesired toxicological effects. Non-limiting examples of such a salt are (a) acid addition salts formed with inorganic acids (e.g., hydrochloric acid, hydrobromic acid, sulphuric acid, phosphoric acid, nitric acid, and the like), and salts formed with organic acids (such as e.g. acetic acid, oxalic acid, tartaric acid, succinic acid, malic acid, ascorbic acid, benzoic acid, tannic acid, palmitic acid, polyglutamic acid, naphthalene sulphonic acid, naphthalene disulphonic acid, polygalacturonic acid and the like); (b) base additional salts formed with metal cations such as zinc, calcium, bismuth, barium, magnesium, aluminum, copper, cobalt, nickel, cadmium, sodium, potassium and the like, or with a cation formed from ammonia, N,N-dibenzylethylenediamine, D-glucosamine, tetraethylammonium or ethylenediamine; or (c) combinations of (a) and (b); e.g. a zinc tannate or the like.

Pharmaceutically acceptable salts include salts of compounds of the invention that are safe and effective for use in mammals and that possess a desired therapeutic activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention may form pharmaceutically acceptable salts with various amino acids. Suitable base salts include, but are not limited to, aluminum, calcium, lithium, magnesium, potassium, sodium, zinc, and diethanolamine salts. For additional information on some pharmaceutically acceptable salts that can be used to practice the invention please reviews such as Berge, et al., 66 J. PHARM. SCI. 1-19 (1977), Haynes, et al, J. Pharma. Sci., Vol. 94, No. 10, October 2005, pgs. 2111-2120 and See, e.g., P. Stahl, et al., HANDBOOK OF PHARMACEUTICAL SALTS: PROPERTIES, SELECTION AND USE, (VCHA/Wiley-VCH, 2002); S. M. Berge, et al., “Pharmaceutical Salts,” Journal of Pharmaceutical Sciences, Vol. 66, No. 1, January 1977.

Pharmaceutical formulation: The compounds of the invention and their salts may be formulated as pharmaceutical compositions for administration. Such pharmaceutical compositions and processes for making the same are known in the art for both humans and non-human mammals. See, e.g., REMINGTON: THE SCIENCE AND PRACTICE OF PHARMACY, (A. Gennaro, et al., eds., 19^{th} ed., Mack Publishing Co., 1995). Formulations can be administered through various means, including oral administration, parenteral administration such as injection (intramuscular, subcutaneous, intravenous, intraperitoneal) or the like; transdermal administration such as dipping, spray, bathing, washing, pouring-on and spotting-on, and dusting, or the like. Additional active ingredients may be included in the formulation containing a compound of the invention or a salt thereof.

The pharmaceutical formulations of the present invention include those suitable for oral, parenteral (including subcutaneous, intradermal, intramuscular and intravenous) and rectal administration. The formulations may be presented in unit dosage form and may be prepared by any of the methods well known in the art of pharmacy. All methods include the step of bringing into association the active ingredient, i.e., the compound or salt of the present invention, with the carrier. In general, the formulations are prepared by uniformly and intimately bringing into association the active ingredient with a liquid carrier or, a finely divided solid carrier or both, and then, if necessary, forming the associated mixture into the desired formulation.

The pharmaceutical formulations of the present invention suitable for oral administration may be presented as discrete units, such as a capsule, cachet, tablet, or lozenge, each containing a predetermined amount of the active ingredient; as a powder or granules; as a solution or a suspension in an aqueous liquid or non-aqueous liquid such as a syrup, elixir or a draught, or as an oil-in-water liquid emulsion or a water-in-oil liquid emulsion. The formulation may also be a bolus, electuary or paste.

The pharmaceutical formulations of the present invention suitable for parenteral administration include aqueous and non-aqueous sterile injection solutions, and may also include an antioxidant, buffer, a bacteriostat and a solution which renders the composition isotonic with the blood of the recipient, and aqueous and non-aqueous sterile suspensions which may contain, for example, a suspending agent and a thickening agent. The formulations may be presented in a single unit-dose or multi-dose containers, and may be stored in a lyophilized condition requiring the addition of a sterile liquid carrier prior to use.

Pharmaceutically acceptable carrier: Pharmaceutically acceptable carrier, unless stated or implied otherwise, is used herein to describe any ingredient other than the active component(s) that maybe included in a formulation. The choice of carrier will to a large extent depend on factors such as the particular mode of administration, the effect of the carrier on solubility and stability, and the nature of the dosage form.

A tablet may be made by compressing or moulding the active ingredient with the pharmaceutically acceptable carrier. Compressed tablets may be prepared by compressing in a suitable machine the active ingredient in a free-flowing form, such as a powder or granules, in admixture with, for example, a binding agent, an inert diluent, a lubricating agent, a disintegrating and/or a surface active agent. Moulded tablets may be prepared by moulding in a suitable machine a mixture of the powdered active ingredient moistened with an inert liquid diluent. The tablets may optionally be coated or scored and may be formulated so as to provide slow or controlled release of the active ingredient.

A “selective” Rho kinase 1 (ROCK1) inhibitor is one that has at least 2, 5, 10, 20, 50, 100, or 200 fold greater inhibitory activity (for example, as determined by calculation of IC₅₀, Kᵢ, or other measure of affinity or effect) for a particular isozyme of ROCK1 compared to other members of the ROCK family.

As used herein, “Rho kinase (ROCK) inhibitors” include, but are not limited to, Fasudil, H-1152, Ripasudil, RKI-1447, Y-27632, GSK429286A, Y-30141, and nitric oxide (NO), or a derivative and/or a pharmaceutical salt thereof.

As used herein, “Rho inhibitors” include, but are not limited to, rhosin, Y16, and C3 Transferases, or a derivative and/or a pharmaceutical salt thereof.

As used herein, “anthracyclines (ANTs)” include, but are not limited to, daunorubicin, doxorubicin, epirubicin, idarubicin, pixantrone, sabarubicin, and valrubicin, or a derivative and/or a pharmaceutical salt thereof.

As used herein, “tyrosine kinase inhibitors (TKIs)” include, but are not limited to, imatinib, dasatinib, nilotinib, bosutinib, ponatinib, lapatinib, adavosertib, gefitinib, erlotinib, toceranib, sorafenib, nilotinib, afatinib, axitinib, cabozantinib, osimertinib, lenvatinib, midostaurin, neratinib, regorafenib, and vandetanib, or a derivative and/or a pharmaceutical salt thereof.

As used herein, “statins” include, but are not limited to, atorvastatin, fluvastatin, lovastatin, pitavastatin, pravastatin, rosuvastatin, simvastatin, and ezetimibe/simvastatin, or a derivative and/or a pharmaceutical salt thereof.

As used herein, “therapeutic regime” and/or “therapeutic regimens” include, but are not limited to, surgery, chemotherapy, radiation therapy, bone marrow transplant, targeted therapy, precision medicine, immunotherapy, stem cell transplant, hyperthermia, photodynamic therapy, blood product donation and transfusion, Light Amplification by Stimulated Emission of Radiation (LASER) in cancer treatment, DNA-damaging radiotherapy, endocrine therapy, and hormone therapy.

As used herein, “therapeutic agents” include, but are not limited to, anti-cancer agents, antibiotics, chemotherapy agents, antileukemic agents, anthracyclines (ANTs), and tyrosine kinase inhibitors (TKIs).

As used herein, “cancer” includes, but is not limited to, bone cancer, brain cancer, breast cancer, endocrine cancer, gastrointestinal cancer, gynaecologic cancer, head and neck cancer, hematologic cancer, lung cancer, prostate cancer, renal cell carcinoma, skin cancer, urologic cancer, rare cancers, hematologic malignancies, lymphoma, leukemia, acute lymphoblastic leukemia (ALL), acute myeloid leukemia (AML), chronic lymphocytic leukemia (CLL), and chronic myeloid leukemia (CML).

The term, “synergism” or “synergy” refers to an interaction of two or more factors such that the effect when combined is greater than the predicted effect based on the response of each factor applied separately.

Treatment of AML patients with anthracycline (e.g., DOX) increases remission rate and survival. However, the dose-related toxic side effects of DOX remain a major concern. While, reduction of the cumulative dose of DOX remains the most valid option to reduce the risk of DOX induced CTX; 6-18% of cancer patients including AML patients suffer from clinical or sub-clinical manifestation of heart failure within 9 years of DOX treatment. Regardless of the severe CTX, ANTs such as DOX are irreplaceable in cancer therapeutic schemes. Thus, identification of strategies to mitigate DOX induced CTX are critical for better outcomes in AML patients to reduce the incidence of relapse and toxic side effects due to this treatment.

Various mechanism(s) have been proposed to explain DOX induced CTX during cancer treatment; they include, but are not limited to free radical-induced oxidative stress, damage to nuclear DNA, dysregulation of calcium handling and cellular contractility, suppression of transcription factors that regulate cell survival and sarcomere protein synthesis, disruption of sarcomere stability, and mitochondrial dysfunction in cardiomyocytes. Most of these cellular events eventually contribute to cardiomyocyte death. There is growing evidence to suggest that cardiomyocyte apoptosis and impaired autophagic function are dominant contributors of DOX-induced cardiomyopathy. DOX-induced cardiac injury is associated with dysregulation in autophagic function and causes an over-activation of autophagy initiation due to increased cellular damage while preventing autophagy completion due to deleterious effects on lysosomes, which results in the accumulation of un-degraded protein aggregates or damaged organelles. Attenuating autophagic dysregulation in DOX-treated hearts is potentially an attractive strategy to prevent or mitigate DOX-induced cardiomyopathy in AML patients.

Clearly, development of chemotherapeutic agents such as DOX has resulted in prolonged survival outcomes for AML patients. However, in the shadow of these successes, severe cardiovascular toxicities have been documented, including with newer therapies called, “targeted therapies”, which were initially considered to have minimal cardiovascular adverse effects.

Major concerns have arisen over a number of alarming vascular adverse events (VAEs) due to second and third-generation BCR-ABL TKIs. Briefly, TKIs have revolutionized the treatment of Ph+ ALL and CML, two blood cancers due to chromosomal translocation encoding the oncoprotein BCR-ABL. This translocation results in constitutive activation of BCR-ABL tyrosine kinase signaling, which drives aberrant cell growth and survival. In 2001, Imatinib was the first TKI approved for the treatment of CML and it showed phenomenal efficacy in CML patients. However, due to lack of response in some patients and the development of acquired drug resistance in 20 to 30% of patients, two additional potent second generation TKIs (e.g., dasatinib and nilotinib) were developed. Although these TKIs demonstrated improved patient outcomes, resistance occurred in a subset of patients harboring BCR-ABL mutations, particularly the T315I mutant.

Ponatinib, a novel multi-targeted TKI and potent pan-ABL inhibitor, was approved for the treatment of Ph⁺ ALL and CML patients who were resistant or intolerant to prior TKI therapy. As compared with its predecessors, Ponatinib was more potent c-ABL inhibitor and could also inhibit the activity of the BCR-ABL T315I mutation, which is present in about 20% of treatment-resistant CML patients. Clinical studies reported that a 54% major cytogenetic response rate was observed in CML patients treated with Ponatinib. Nonetheless, Ponatinib's clinical efficacy has been tempered by reports of CTX in patients. For instance, Ponatinib's package insert describes the risk of cardiac-specific toxicities such as myocardial infarction (MI), severe congestive heart failure (HF), and cardiac arrhythmias (Ariad, 2013). In addition, the risk of life-threatening narrowing of blood vessels resulted in Ponatinib's temporarily withdrawal (FDA, 2013), although it was later returned with enhanced warnings of these safety concerns. Ponatinib and Nilotinib are also associated with increased vascular injury. Ponatinib is an example of a growing number of multitargeted oncology therapies that have shown unexpected CTX in patients. Although these drugs have transformed leukemia treatment and led to increased patient survival, they often induce toxicity due to inhibition of critical targets and pathways shared by both leukemic and cardiac cells.

The recent recognition of adverse cardiovascular impact of TKI therapy, the longer duration of TKI therapy (some CML patients have been on TKIs for over a decade), and the longer lifespans of CML/ALL survivors has brought about tremendous urgency to recognize, define and mitigate cardiovascular risk in these patients. While CTX resulting in heart failure is a well-recognized complication of certain traditional chemotherapeutics, CTX is now recognized as a side effect of TKI treatments.

Rho associated kinase (ROCKs) are central regulators of the actin cytoskeleton downstream of the small GTPase RhoA. The two ROCK isoforms, ROCK1 and ROCK2, are highly homologous with an overall amino acid sequence identity of 65%. In this disclosure, ROCK1 was found to be a key molecule in mediating apoptotic signalling in cardiomyocytes under pressure overload and in genetically-induced pathological cardiac hypertrophy. Using mouse embryonic fibroblasts as an in vitro system, it was observed that ROCK1 deficiency has a unique protective benefit of preserving actin cytoskeleton stability, which acts additively with antioxidant treatment to suppress excessive production of doxorubicin-induced reactive oxygen species and apoptosis. Based on these in vitro observations, in vivo role of ROCK1 in mediating DOX cardiomyopathy, including its impact on autophagy function was also examined.

Embodiments of the present invention is further illustrated by the following non-limiting examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention or the scope of the appended claims.

### EXAMPLES

### Example 1: Deficiency of ROCK1 Protects Mice from DOX-Induced CTX

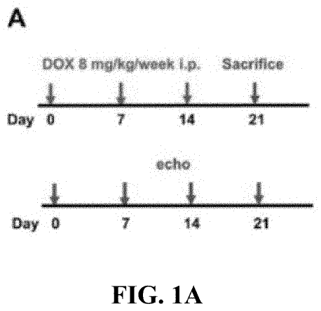

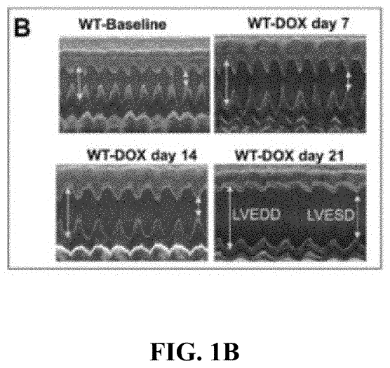

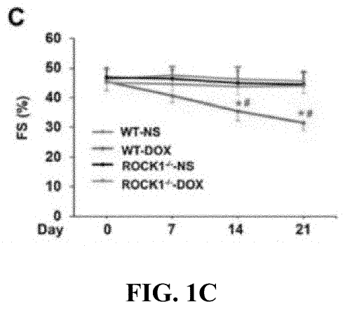

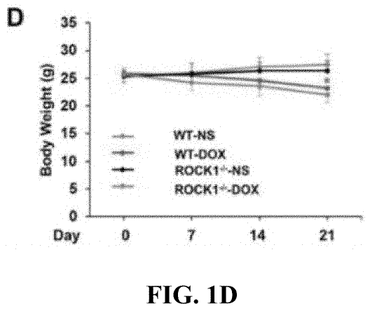

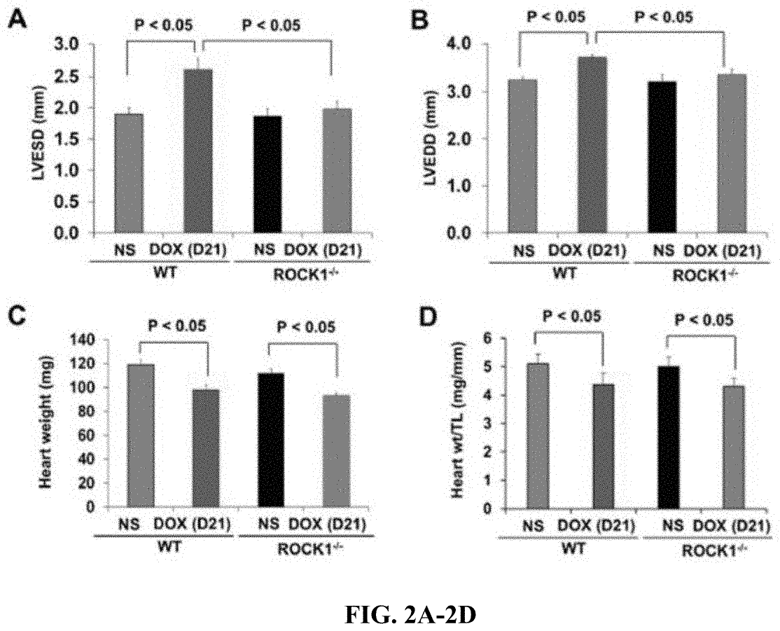

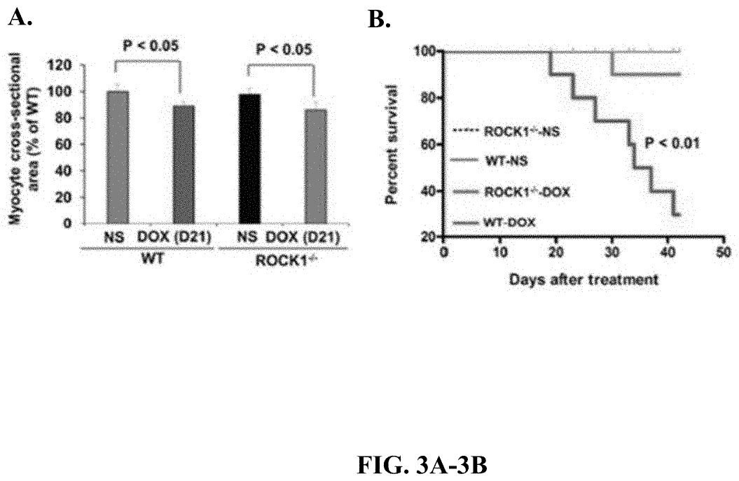

The generation of ROCK1 deficient mice have been previously reported. (See Zhang, Y. M., et al. Targeted deletion of ROCK1 protects the heart against pressure overload by inhibiting reactive fibrosis. FASEB J 20, 916-925 (2006)). To assess if a deficiency of ROCK1 protects the mice from DOX induced CTX, control (WT) and ROCK1^{-/-} mice were injected with 8 mg/kg of DOX or normal saline (NS) once weekly for 3 consecutive weeks, for a cumulative dose of 24 mg/kg (FIG. 1A). This treatment is based on the previously described approach, which causes progressive cardiac dysfunction within three weeks after the initial injection, a scenario that mimics early-onset of DOX-induced CTX in humans. Referring now to FIG. 1B, echocardiograms revealed that DOX treatment resulted in reproducible and progressive left ventricular (LV) dilation in WT mice as evidenced by increased end systolic dimension (LVESD) and end diastolic dimension (LVEDD) over 3 weeks after initiation of the treatment. Consistent with cardiac dilation, LV contractile function (as measured by LV fractional shortening, FS) was reduced in DOX-treated WT mice (FIGS. 1C, 2A, 2B). DOX had a significant impact on body weight (FIG. 1D), heart weight (FIGS. 2C, 2D), and cardiomyocyte size (FIG. 3A). For subsequent studies of phenotype, day 21 after the initial injection was used in part because no significant animal death was detected in WT mice (FIG. 3B).

To elucidate the role of ROCK1 in DOX induced CTX, ROCK1^{-/-} mice were treated with DOX in parallel to the WT mice. Refering now to FIGS. 1 and 2, in contrast to the WT mice, DOX-induced LV dilation was blocked in ROCK1^{-/-} mice, and cardiac function was preserved over 3 weeks after initiation of the treatment (FIGS. 1C, 2A, 2B). DOX had a similar impact on body weight (FIG. 1D), heart weight (FIGS. 2C, 2D), and cardiomyocyte size (FIG. 3A) in ROCK1^{-/-} mice compared to WT mice, indicating that the protective effects of ROCK1 deletion are not mediated through the inhibition of weight loss and cardiomyocyte atrophy. Referring now to FIG. 3B, Kaplan-Meier survival curves over 6 weeks after the initial DOX injection indicated that the mortality rate was significantly lower in the treated ROCK1^{-/-} group (about 10%) than that in the treated WT group (about 70%). In addition, all WT mice treated with DOX died within 3 months, whereas the mortality rate of ROCK1 deficient mice treated with DOX reached to 55% at 3 months and to 70% at 6 months. These results support that ROCK1 deficiency reduces not only CTX but also systemic toxicity caused by DOX.

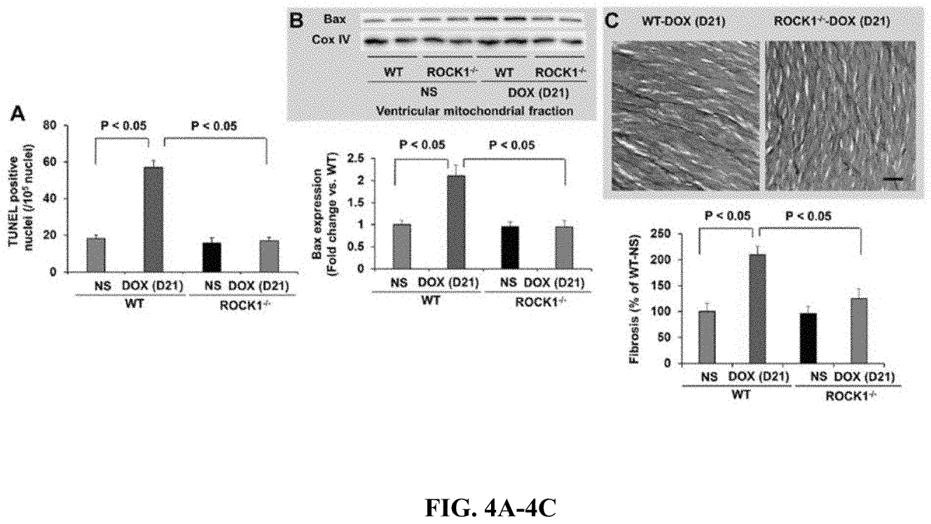

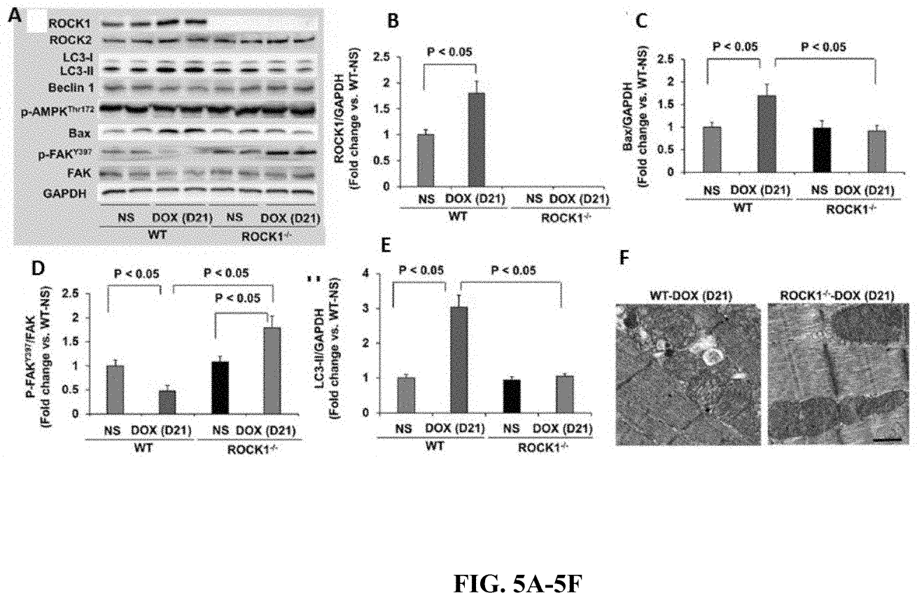

Since DOX treatment induces significant increases in cardiomyocyte and non-cardiomyocyte apoptosis, apoptosis by TUNEL staining were measured. Referring now to FIG. 4, the number of TUNEL positive cardiac cells was significantly increased in DOX-treated WT mouse hearts (FIG. 4A), and was associated with increased mitochondrial translocation of Bax (FIG. 4B). Cardiac fibrosis was also increased in DOX-treated WT mice (FIG. 4C). However, these characteristics of DOX-induced CTX were effectively blocked in ROCK1^{-/-} mice (FIGS. 4A-C). Referring now to FIG. 5, molecular analysis also revealed increased ROCK1 expression in DOX-treated WT mice, associated with increased Bax levels and decreased focal adhesion kinase (FAK) phosphorylation, further supporting a role of ROCK1 in DOX-induced CTX. Given these observations, autophagy in these mice were examined. During autophagy, the cytosolic form of microtubule-associated protein 1 light chain 3 (LC3-I) is conjugated to phosphatidylethanolamine to form LC3-II, which is recruited to autophagosomal membranes. Referring now to FIGS. 5A and 5E, the levels of LC3-II were increased by about 3-fold in DOX-treated WT hearts but not in ROCK1^{-/-} hearts, suggesting that ROCK1 deletion can reduce and/or prevent DOX-induced dysregulation of autophagy. Consistent with this notion, transmission electron microscopy revealed more accumulation of autophagosomes in DOX-treated WT mouse hearts than in ROCK1^{-/-} hearts (FIG. 5F). Moreover, the increased LC3-II accumulation was not accompanied by increased levels of Beclin 1 and AMP-activated protein kinase (AMPK) phosphorylation (FIG. 5A), suggesting that the accumulation of autophagosomes was not due to the activation of either AMPK-mediated autophagy or a general up-regulation of the autophagy system.

### Example 2: Cardiomyocyte-Specific ROCK1 Knockout Mice are Also Protected from DOX-Induced CTX

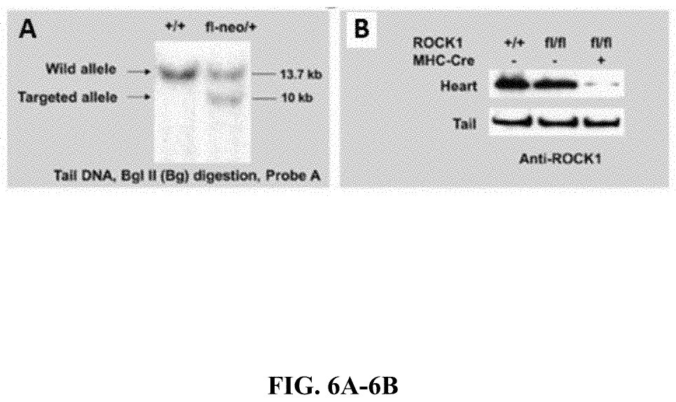

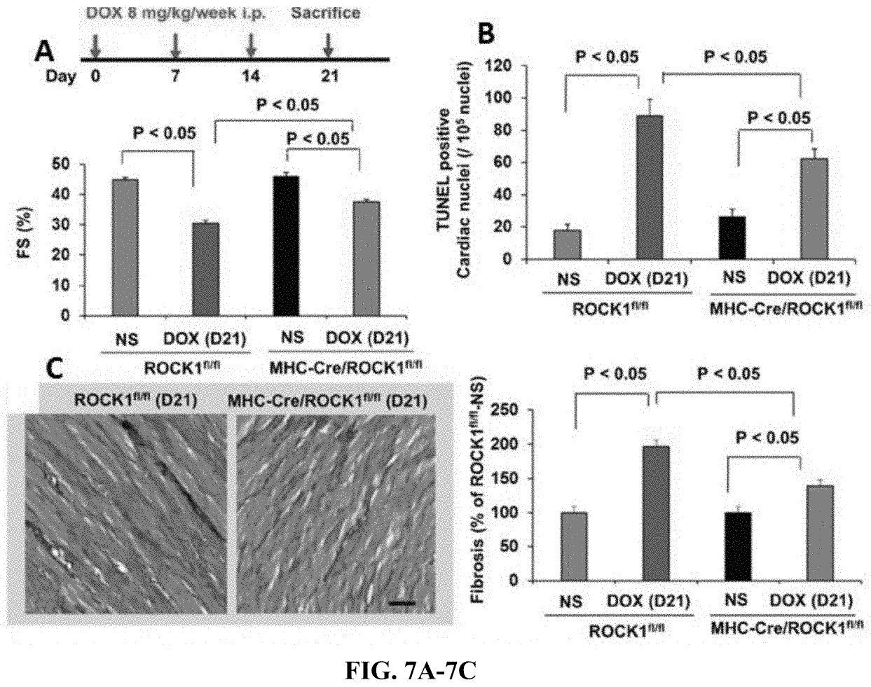

To determine if ROCK1 in cardiomyocytes has a role in DOX-induced CTX, cardiomyocyte-specific ROCK1 knockout mice were generated. In myosin heavy chain (MHC)-Cre/ROCK1^{fl/fl} mice, the ROCK1 protein is truncated only in cardiomyocytes from residue 137 to the end of the protein (FIGS. 6A, 6B). Similar to ROCK1^{-/-} mice in which the ROCK1 protein is also truncated from residue 180 to the end of the protein in all cells, the conditional targeting approach results in the removal of a large portion of the kinase domain and amino acids from the coiled-coil and the PH domains. It was noted that MHC-Cre alone had no significant cardiotoxic effects at baseline as no significant morphological or molecular differences in apoptosis and autophagy markers were observed in MHC-Cre mice compared to WT mice (data not shown). Referring now to FIG. 7, the cardiac response to DOX were examined in MHC-Cre/ROCK1^{fl/fl} mice (e.g., 8 to 9 weeks old) and ROCK1^{fl/fl} mice. These mice were subjected to a three-injection treatment regime (FIG. 7A). Similar to the findings in WT mice, DOX also induced cardiac dysfunction, increased TUNEL positivity and cardiac fibrosis in ROCK1^{fl/fl} hearts. However, these cardiotoxic events were significantly reduced in MHC-Cre/ROCK1^{fl/fl} mice, supporting that ROCK1 in cardiomyocytes contributes to DOX-induced CTX (FIGS. 7A-C).

### Example 3: Constitutive Activation of ROCK in BCR-ABL and FLT3-ITD (FLT3N51) Bearing Cells

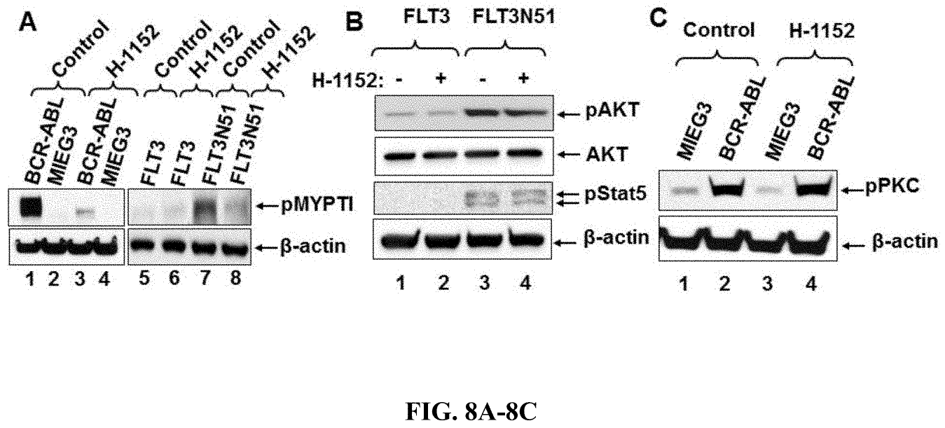

Cells expressing the BCR-ABL or the FLT3-ITD mutation (found in close to 30% of all AML patients with normal cytogenetics and associated with poor overall survival) were starved and ROCK activity was analyzed by assessing the phosphorylation of its substrate myosin phosphatase (MYPT1). This was done in the presence or absence of a highly specific and potent ROCK inhibitor H-1152. Similar results were observed with clinically approved ROCK inhibitor, Fasudil. Referring now to FIG. 8A, constitutive activation of ROCK was observed only in oncogene bearing cells, but not in cells bearing the WT receptor or the empty vector. Further, H-1152 treatment rapidly inhibited ROCK activity in BCR-ABL and FLT3-ITD bearing cells. Referring now to FIGS. 8B and 8C, H-1152 treatment had no effect on the activation of AKT, Stat5 or PKC in oncogene bearing cells. These results suggest that BCR-ABL and FLT3-ITD induce constitutive ROCK activation, which is inhibited by H-1152 and Fasudil.

### Example 4: ROCK Inhibitors Suppress the Constitutive Growth of BCR-ABL, BCR-ABLT315I and FLT3-ITD Bearing Cells

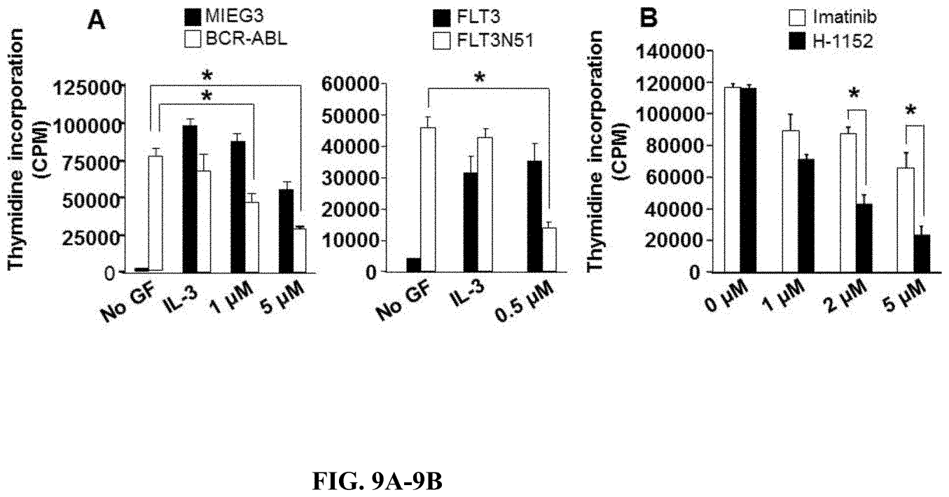

Referring now to FIG. 9, cells bearing empty vector or FLT3 showed minimal thymidine incorporation in the absence of growth factors (FIG. 9A). IL-3 enhances the growth of these cells. In contrast, cells expressing BCR-ABL, FLT3-ITD or BCR-ABL^{T315I} showed constitutive growth in the absence of growth factors, which was repressed by H-1152 in a dose dependent manner. H-1152 treatment of cells bearing vector or FLT3 in the presence of IL-3 showed minimal suppression in proliferation (FIG. 9A). Other ROCK inhibitors Fasudil and Y27632 similarly repress the growth of cells bearing BCR-ABL or FLT3-ITD (data not shown). Moreover, the growth of imatinib resistant mutant of BCR-ABL^{T315I} was also suppressed in the presence of H-1152 (FIG. 9B). In order to illustrate the imatinib resistance, very high dose of imatinib were used in these cells. These results suggest that ROCK may play a prominent role in supporting the growth of oncogene bearing cells (in particular a form of BCR-ABL mutant, BCR-ABL^{T315I}, which is resistant to imatinib treatment) but only a modest role in supporting the growth of normal hematopoietic cells bearing non-oncogene. This could provide a new approach for treating AML and CML patients.

### Example 5: ROCK Inhibitor Suppresses the Growth of Primary FLT3ITD Positive Cells from AML Patients

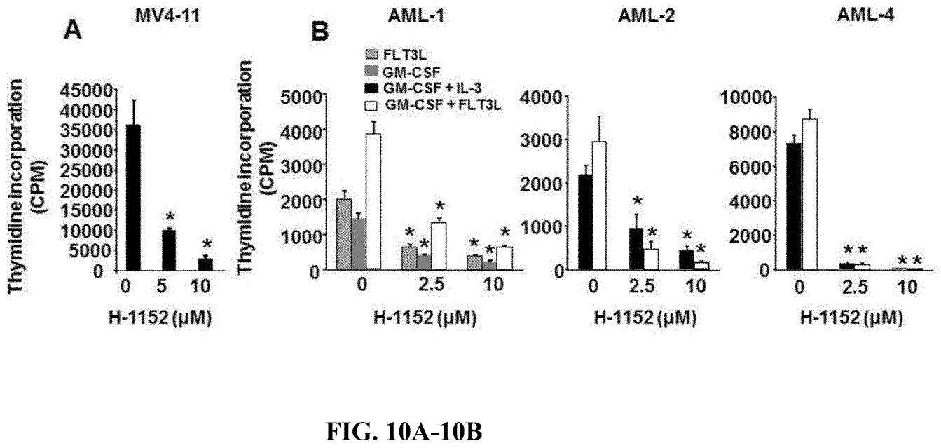

Referring now to FIG. 10, 16 primary AML patient derived cells positive for FLT3-ITD expression were examined and a cell line bearing FLT3-ITD (MV4-11) was used as a positive control. Treatment with H-1152 showed a dose dependent reduction in the growth of all of the primary AML cells (FIG. 10; results from 3 representative samples are shown).

### Example 6: ROCK Inhibitor Suppresses the Growth of Primary BCR-ABL Positive Cells from CML Patients

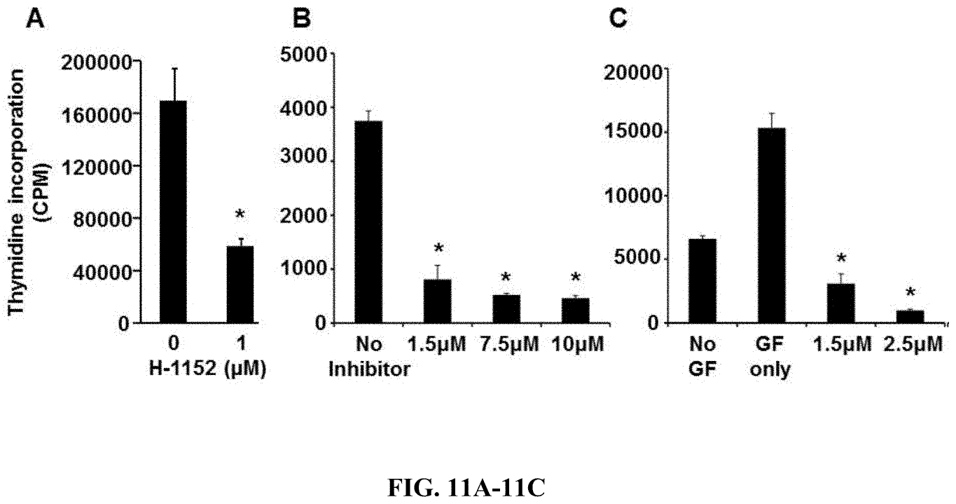

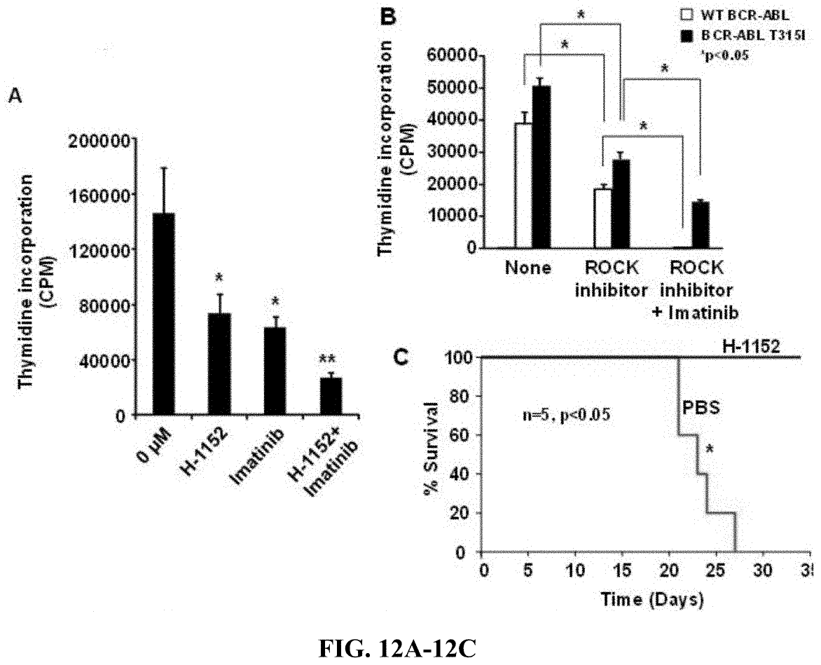

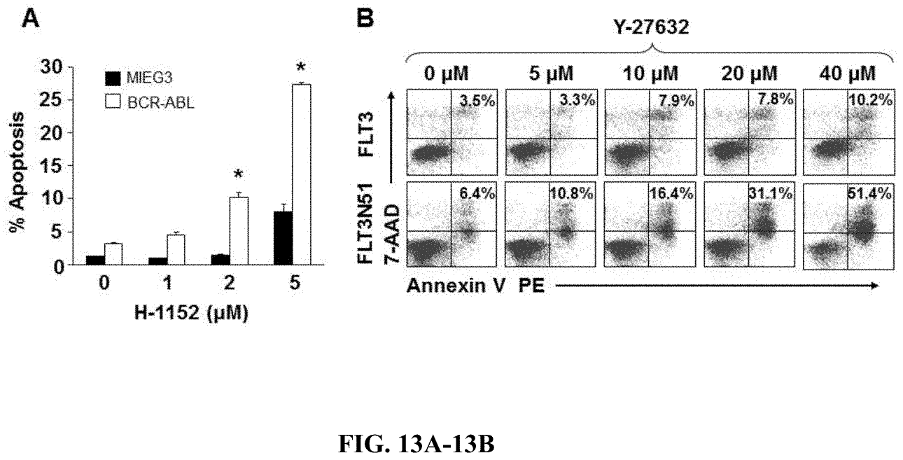

Referring now to FIG. 11, primary CML patient derived cells were examined and a human cell line bearing BCR-ABL (K562) was used as a positive control. H-1152 showed a dose dependent reduction in the growth of all of these cells. Referring now to FIG. 12, a synergic effect between the ROCK inhibitor and imatinib in reducing the growth of BCR-ABL expressing cells was observed (FIG. 12A). Importantly, the combination of ROCK inhibitor and imatinib also showed efficacy in cells expressing the imatinib resistant form of BCR-ABL (BCR-ABL^{T315I}; FIG. 12B) and prolonged the overall survival of mice bearing cells expressing BCR-ABL T315I and treated with the ROCK inhibitor (FIG. 12C). The reduction in ROCK inactivation induced growth in leukemic cells is due to enhanced apoptosis (FIG. 13). Taken together, using primary patient derived cells, as well as transplant studies, the results provide anti-proliferative/survival activity of ROCK inhibitors against oncogenic mutations associated with AML and CML. Accordingly, ROCK inhibitor, Fasudil, can be used to modulate/treat BCR-ABL-induced CML in vivo and can also be used with imatinib to further enhance survival of mice bearing WT, BCR-ABL, and/or BCR-ABL^{T315I}, and further, protection of these mice from CTX.

These and additional conditional knockout models of ROCK1 deficiency in noncardiomyocyte cardiac cells including VSMCs and ECs, are being utilized in conjunction with systemic ROCK1 knockout mice in the context of AML to assess the relative contribution of ROCK1 in mitigating DOX-induced CTX. The main objective here is to mitigate CTX in the context of preserving and/or enhancing anti-leukemic potential. Thus, the cardioprotective role of ROCK1 inactivation can be clinically meaningful if examined in the context of leukemia and other cancers. Data disclosed herein further demonstrate that inactivation of ROCK(s) in leukemic/cancer cells (e.g., AML and CML cells) can inhibit growth and induce apoptosis of these cells while sparing normal cells. While not wishing to be bound by this theory, the differences in functional outcomes due to ROCK1 inactivation in normal vs. tumor cells can be due to variations in anchorage-dependency, cytoskeleton organization and interacting partners in individual cell types. In this disclosure, a molecule, in the form of ROCK1, have been identified where its inactivation in cardiomyocytes and in non-cardiomyocyte cardiac cells preserves function, while its inactivation in leukemic cells induces cell death.

### Example 7: DOX-Induced ROCK Activation can Lead to the Development of CTX During AML Treatment Through Increased Apoptosis and Impaired Autophagy

The impact of genetic modification of ROCK1 globally or specifically in cardiomyocytes, vascular smooth muscle cells and endothelial cells will be measured on the development of CTX including fibrosis, apoptosis and autophagy dysregulation in a mouse model of human AML treated with clinically relevant doses of DOX to enhance its anti-leukemic activity.

CTX is a major concern in treating AML patients. AML patients, both young and old with additional co-modalities including pre-existing heart conditions are especially susceptible and vulnerable to chemotherapy-induced CTX. Unfortunately, almost all AML patients treated with chemotherapy such as DOX will likely relapse. Studies suggest that lack of dose escalation to completely eradicate AML blasts and leukemia stem cells is not always feasible in these patients in part due to the associated CTX, and the CTX plays a major role in relapse. Thus, duration and dose of DOX administration in AML patients can be a motility- and mortality-determining factor in both children and adults. Data disclosed herein suggests that global ablation of ROCK1 protects the heart from DOX-induced CTX. The data further suggest that the protection from DOX-induced CTX is partly attributable to the loss of ROCK1 expression in cardiomyocytes. In addition to DOX induced increased expression of ROCK1 in cardiomyocytes, its increase in VSMCs as well as in ECs can also contribute to DOX-induced CTX and increased apoptosis. The role of ROCK1 induced autophagy regulation in cardiomyocytes will be determined and the role of DOX induced ROCK1 expression in VSMCs and ECs will be assessed, for example, in AML model driven by a combination of genetic and epigenetic mutations.

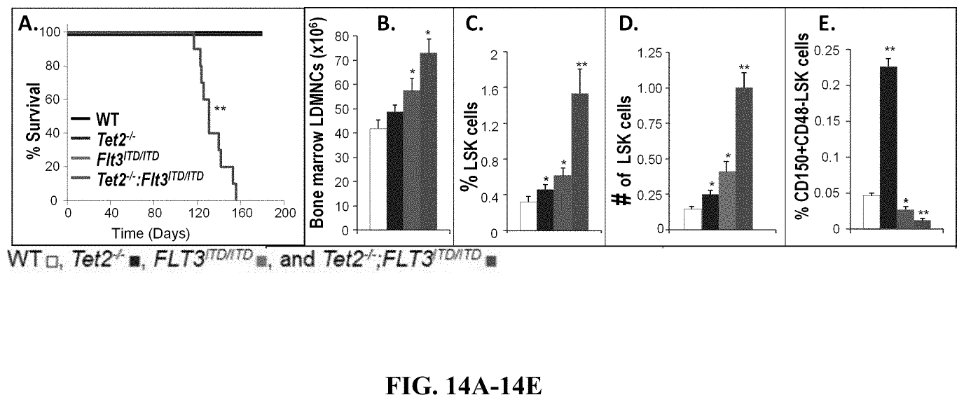

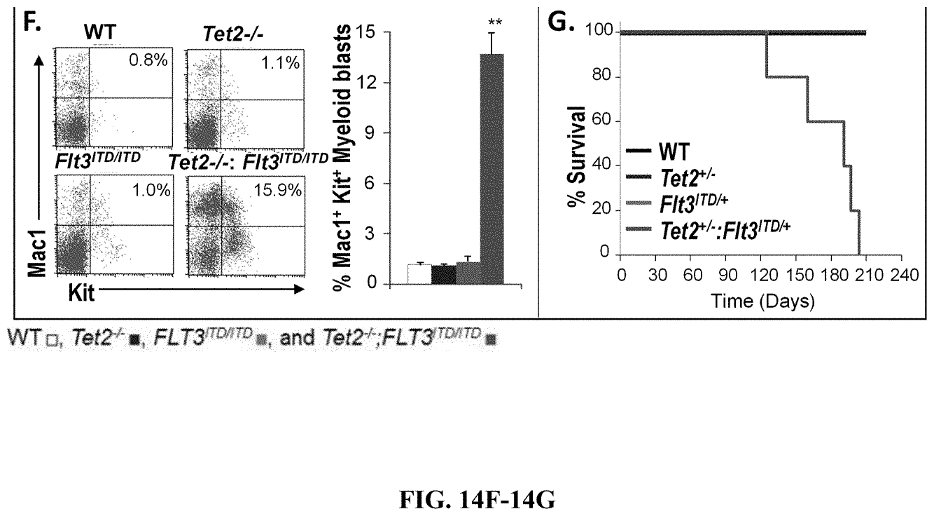

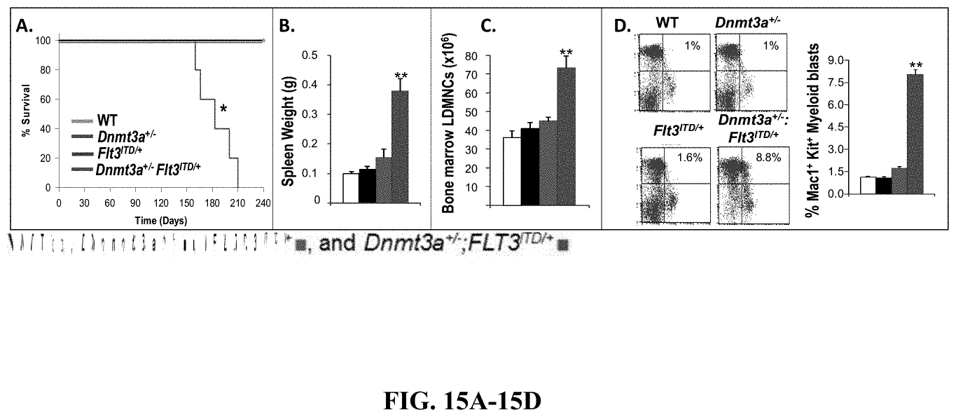

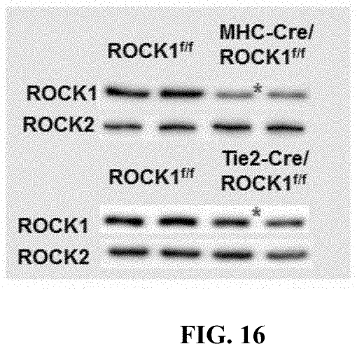

Two mouse AML models that mimic all the cardinal features of human AML were generated. Loss of epigenetic regulator TET2 or DNMT3A is considered an early genetic event that occurs in pre-leukemic stem cells (pre-LSCs) in the human bone marrow while FLT3-ITD mutation is acquired later on. To understand how the FLT3-ITD oncogene cooperates with epigenetic regulators and whether this cooperation can promote the switch from a pre-LSC to a leukemia stem cell (LSC) state, mice expressing FLT3-ITD driven by its endogenous promoter with concomitant loss of Tet2 or Dnmt3a were generated. None of the mice with mutations in single gene show signs of AML; however, when these mice were inter-crossed, they all develop AML with an early onset and complete penetrance. The disease is fatal and fully transplantable in lethally irradiated secondary hosts (FIGS. 14 and 15). Compound mutant, Tet2^{-/-}FLT3^{ITD/+} mice consistently demonstrate increase in peripheral blood WBC counts with increase in myeloid cells, splenomegaly, increased BM cellularity and presence of myeloid blasts (FIG. 14 and data not shown). Similar phenotype was observed in Dnmt3a^{+/-}FLT3^{ITD/+} mice (FIG. 15). Taken together, loss of Tet2 or Dnmt3a in the presence of FLT3-ITD cooperates to alter proliferation and differentiation of stem cells, leading to the development of AML. Additionally, the leukemia-initiating potential in these mice has been demonstrated to reside in the lineage negative, Sca1 positive, and KIT positive (LSK) fraction of the bone marrow. It has been reported that FLT3-ITD, DNMT3A or TET2 mutations are found in about 30% of human AML patients with normal cytogenetics and are associated with poor overall survival. These AML mouse models will be used to examine anti-leukemic efficacy of DOX and protection from CTX in the systemic loss of ROCK1 or tissue specific loss of ROCK1. lineage negative Sca1⁺KIT⁺ (LSK; population that contains the leukemia stem cells) will be purified from the bone marrow of these AML mice. LSK cells will be transplated into mice of 5 distinct genotypes lacking ROCK1 either systemically or in a tissue specific manner. These mice will function as recipients of LSK AML donor cells. The following 5 genotypes will function as recipients of AML LSK cells: 1) WT; 2) ROCK1^{-/-} (systemic ablation); 3) ROCK1^{fl/fl}-MHC-Cre (cardiac specific ablation of ROCK1); 4) ROCK1^{fl/fl}-SM22α (vascular smooth muscle cell specific ablation of ROCK1); and 5) ROCK1^{fl/fl}-Tie2-Cre (vascular endothelial cell specific ablation of ROCK1). Referring now to FIG. 16, ROCK1^{fl/fl}-SM22α and ROCK1^{fl/fl}-Tie2-Cre mice have been generated. All mice are viable.

Three different sources of donor cells will be utilized: 1) WT LSK cells; 2) Tet2^{-/-}FLT3^{ITD/+} LSK cells; and 3) Dnmt3a^{-/-}FLT3^{ITD/+} LSK cells. Lethally irradiated CD45.1⁺ (BoyJ) recipient mice of the above described 5 genotypes will be transplanted with CD45.2⁺WT, Tet2^{-/-}FLT3^{ITD/+} or Dnmt3a^{-/-}FLT3^{TID/+} leukemic cells along with CD45.1⁺ normal cells in 1:1 ratio pooled from 2-3 mice of either gender at 4 months of age. 2 months posttransplant, the development of AML in these mice will be assessed by analysing peripheral blood. Mice will be randomly assigned into two groups: 1) vehicle; and 2) intraperitoneal injection (IP) of DOX 8 mg/kg/week for 3 weeks. This treatment is based on the previously described approach. This type of treatment results in complete remission of AML in these mice, loss of AML blasts and reduction in the number of leukemia stem cells. Preliminary results show that this drug regimen results in severe CTX including dysfunction in WT mice (data not shown). This includes progressive cardiac dysfunction within three weeks after the initial injection, a scenario that mimics early-onset DOX-induced CTX in humans. Alternatively, mice will be subjected to 5 IP injections of DOX (5 mg/Kg) with one-week intervals for a cumulative dose of 25 mg/kg over a period of 5 weeks. This will allow for a longer survival of 20 weeks. Relapse of these mice will be monitored closely and DOX will be re-administered and then be compared for CTX.

At the end of a therapy (e.g., 3 or 5 weeks from initial DOX injection), some animals will be euthanized and the effects on differentiated, progenitor and leukemia stem cells in the peripheral blood, spleen and bone marrow will be quantified by flow cytometric analysis including the frequency and numbers of blasts (Kit⁺/Mac-1⁺). Given that the presence of these mutations results in increased number and frequency of granulocyte macrophage progenitors (GMPs), LSKs and MPPs, the extent to which this treatment corrects these phenotypes will be evaluated. Tissues from organs such as spleen, liver, lung will be fixed in formaldehyde for histological evaluation. AML model disclosed herein will allow for simultaneous evaluation of effects of DOX on normal hematopoietic cells (CD45.1) and leukemic cells (CD45.2). These cells can be distinguished on the basis of one antigen difference by capturing these cells using flow cytometry on the basis of surface expression of CD45.1 vs CD45.2. To investigate if the reduction in CD45.1 vs CD45.2 cells after treatment is due to induction of cell death or inhibition of proliferation of leukemic stem cells and blasts, Annexin-V staining and Ki67 expression analysis will be performed, respectively. The remaining cohort of mice will be monitored for survival and disease re-emergence by periodic quantification of peripheral blood counts with phenotypic analysis. The mice will be monitored for at least 4 months. If leukemia stem cells (LSCs) are functionally ablated, then a prolonged disease-free survival will be achieved. In an effort to prove this experimentally, LSK cells (i.e., LSCs) from drug treated mice will be purified at the time of harvest and a secondary transplantation assay will be performed by conducting a limiting dilution assay to assess if DOX treatment has eliminated LSCs.

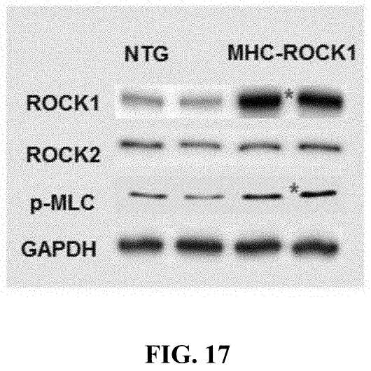

In all five experimental groups described above, cardiac function will be monitored by echocardiograms. Data disclosed herein provides that DOX treatment results in reproducible and progressive LV dilation in WT mice as evidence by increased LVESD and end LVEDD over 3 weeks after initiation of DOX treatment. Consistent with cardiac dilation, LV contractile function is also reduced in these mice. Furthermore, DOX also affects body weight, heart weight and cardiomyocyte size. The changes in ROCK1^{-/-} mice will assessed as well as in mice conditionally lacking ROCK1 in specific cell types in the context of AML. In addition, overall survival of DOX treated mice under these conditions will also be examined. Given the data showing increased apoptosis and dysregulated autophagy in WT cardiomyocytes upon DOX treatment, and its modulation in systemic ROCK1 deficient mice, the mechanism to determine how ROCK1 deficiency in cardiomyocytes, VSMCs and ECs modulates apoptosis and autophagy will further be examined. These studies will be conducted in the context of mice bearing AML and treated with DOX. In addition to examining apoptosis by Annexin V and TUNEL staining in various ROCK1 deficient mice, it will be determined if the change in apoptosis in various ROCK1 KO recipient mice is due to reduced mitochondrial translocation of Bax, which is frequently observed in WT cardiac cells. Given that DOX also induces cardiac fibrosis in WT mice treated with DOX, it will be assessed if loss of ROCK1 blocks or modulates cardiac fibrosis in various conditional knockout of ROCK1. In this disclosure, increased expression of ROCK1 in DOX treated WT mice was observed, which was associated with increased Bax levels and decreased focal adhesion kinase (FAK) phosphorylation, providing further support for a role of ROCK1 in DOX-induced CTX. To directly assess the role of overexpression of ROCK1 in CTX, a ROCK1 cardiac specific transgenic mice have been generated. Referring now to FIG. 17, a significant overexpression of ROCK1 is observed in these transgenic mice compared to controls. A transgenic mouse line with 5-fold overexpression of ROCK1 in cardiomyocytes will be used to transplant AML cells as described above to test if overexpression of ROCK1 induces early death due to enhanced CTX, fibrosis and apoptosis compared to controls when treated with the same dose of DOX as described above. These studies would provide direct evidence for ROCK expression in regulating CTX in the presence of DOX in AML mice.

In addition to DOX induced apoptosis, the data disclosed herein also revealed a novel function of ROCK1 in mitigating DOX induced autophagy dysregulation. The levels of LC3-II were increased by about 3-fold in DOX-treated WT hearts but not in ROCK1^{-/-} hearts, suggesting that ROCK1 deletion prevents the dysregulation of autophagy induced by DOX. This data will be investigated in more detail in the context of AML and in various systemic and conditional ROCK1^{-/-} mice treated with DOX. Molecular analysis will be performed on day 2, 4 and 7 after a 10 mg/kg single dose injection of DOX in ROCK1 deficient mice bearing AML. An increase in the autophagy marker, LC3-II, will be determined. The increase in the level of LC3-II could occur from an overactivation of autophagy initiation or from the prevention of autophagy completion due to deleterious effects on lysosomes. these two possibilities will be distinguished by measuring the accumulation of p62/sequestosome 1 (p62/SQSTM1), another protein marker of autophagy, preceding maximal LC3-II accumulation. Since p62/SQSTM1 is an adaptor protein that is cleared by autophagy, the increased accumulation would suggest that the degradation process of autophagosomes is impaired in DOX treated hearts. Preliminary data provides that ROCK1 deletion abolished DOX-induced increases in LC3-II levels at early time points, suggesting that ROCK1 deletion improves autophagic flux. This will further be assessed in conditional ROCK1 knockout and overexpressing mice.

The phosphorylation levels of Beclin 1 at Thr119 will be examine. ROCK1-mediated Beclin1 phosphorylation promotes the dissociation of Beclin1 from Bcl-2 resulting in increased autophagosome formation. In ROCK1^{-/-} hearts, the levels of p-Beclin1 Thr119 were reduced, suggesting that ROCK1 deletion inhibits Beclin1-mediated autophagosome formation. To examine if ROCK1 deletion can improve autophagic flux, WT and ROCK1^{-/-} mice will be treated with either saline or DOX, and mice will be sacrificed on day 4, 2 hours after an injection of bafilomycin A1 that prevents the fusion of autophagosome with the lysosome and blocks autophagic flux. Bafilomycin A1 injection in control animals should result in an increase in LC3-II levels, reflecting cardiac autophagic flux under basal conditions. Bafilomycin A1 injection in DOX-treated WT mice should show no increase in LC3-II levels, indicating a blockage in autophagic flux. In contrast to WT mice, bafilomycin A1 injection in DOX-treated ROCK1^{-/-} mice will likely result in a significant increase in LC3-II levels, indicating that autophagic flux is maintained in ROCK1^{-/-} mice after doxorubicin treatment. These findings would be consistent with previous studies showing that heterozygous deletion of Beclin 1 is cardio protective to DOX through reducing the initiation of autophagy, which allows for the preservation of autophagic flux in the presence of impaired lysosome function by DOX. similar mechanistic studies will be performed in ROCK1 conditional knockout mice and examined for changes in ROCK1 expression after a single dose of DOX to determine if DOX induces ROCK1 expression in VSMC, ECs and cardiomyocytes. Increases in autophagy markers and an apoptosis marker (Bax) will be assessed in these mice relative to controls and determined if these changes are associated with changes in Beclin1 levels (p-Beclin Thr119 levels) or in phosphorylation of AMPK.

Given that VSMCs and ECs also targets of DOX-induced cell toxicity, in vivo evaluation will be performed by monitoring angiogenesis. Functional coronary microcirculation perfusion will be evaluated through isoflurane induced coronary hyperemia to measure coronary flow reserve (CFR) as previously performed by us in Rnd3 haploinsufficient mice, which exhibit increased ROCK1 activity in the heart. A high frequency ultrasound probe attached to a custom-manufactured micromanipulator will be used for these measurements. Peak left main coronary flow velocity will be recorded at baseline (1% isoflurane) and hyperemic conditions (2.5% isoflurane), respectively. The ratio of hyperemic/baseline coronary velocity will be calculated as the index of CFR. Other measurements will also be performed including quantitation of capillary density by immunostaining of heart sections with anti-CD31, which detect endothelial cells, and quantitation of the levels of the vascular endothelial factor (VEGF) which is involved in development of vessels and stimulates recruitment and proliferation of endothelial cells. DOX treatment will likely result in reduced CFR, reduced capillary density and reduced myocardial VEGF expression, and systemic ROCK1 deletion and conditional ROCK1 deletion will mitigate vascular toxicity. These studies in the context of AML treatment will also be performed.

Because a sex-based difference in leukemia has not been established, studies using mice of both sexes of similar age will be performed. The findings will be corroborated in two different AML models. Recipient mice will be randomly assigned for transplantation purposes and all investigators will be blinded to group assignment. All studies will use C57/BL6 backcrossed mice, including CD45.1 and CD45.2 variants. Systemic ROCK1^{-/-} mice are also carried in FVB background. Donors in the same genetic background will therefore be present.

### Statistical Considerations

The sample size for the mouse experiment is calculated to detect large biological meaningful difference. the CTX between the control and various ROCK1 lacking mice treated with DOX among five groups of recipients will be compared: 1) WT; 2) ROCK1^{-/-} (systemic loss); 3) ROCK1^{fl/fl}-MHC-Cre (cardiac specific loss); 4) ROCK1^{fl/fl}-SM22α (vascular smooth muscle cell specific loss); and 5) ROCK1^{fl/fl}-Tie2-Cre (vascular endothelial cell specific loss). The data will be analyzed using 2-way ANOVA followed by t-test to compare the difference within each recipient group with the multiple comparison adjusted by Tukey's method. A sample size of 10 mice per group will provide 82% power to detect a large effect of 1.3 with a 5% type 1 error.

It is expected that DOX-induced CTX will be improved in ROCK1^{-/-} mice. These findings will support the hypothesis that ROCK1 deficiency not only reduces cardiac toxicity but also systemic toxicity caused by DOX.

While the data supports a role for ROCK1 in CTX, a conditional knockout mouse model of ROCK2 has also been generated and the role of ROCK2 will be investigated. While it has not been observed any increased expression of ROCK2 in ROCK1 knockout mice, if this happens, ROCK1/2 conditional double knockout mice will be utilized as recipients. the role of ROCK1 in protecting human cardiomyocytes, VSMCs, and ECs from DOX induced toxicity will also be examined. Additionally, the protective role of ROCK1 will be assessed in mice undergoing heart failure, which will likely be more susceptible to DOX induced CTX.

### Example 8: Enhanced CTX by TKIs for the Treatment of CML

While not wishing to be bound by this theory, the present disclosure suggests that the enhanced CTX including vascular injury due to TKIs for the treatment of CML and ALL is in part to “on-target” inhibition of Abl1 and Abl2 (Arg) in cardiomyocytes, VSMCs and ECs, resulting in reduced phosphorylation of p190GAP and increased activation of Rho/ROCK pathway. This will further be examined using a model of human CML and ALL lacking ROCK1.

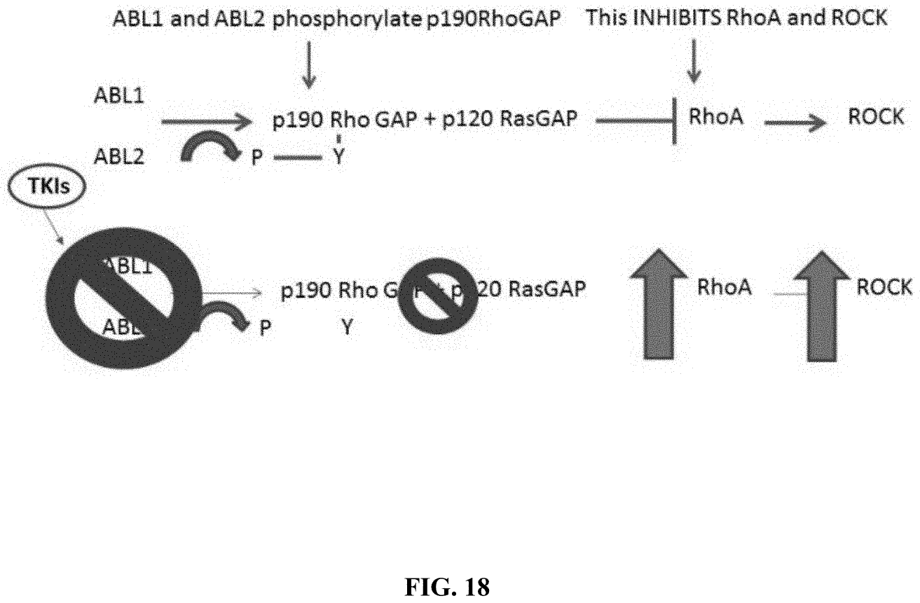

TKIs have changed the outcome of a fatal disease such as CML, into a manageable chronic condition for most patients. Imatinib, the first BCR-ABL TKI, inhibits the ABL kinase. In recent years, newer generations of BCR-ABL kinase inhibitors (dasatinib, nilotinib and ponatinib) have been developed to overcome Imatinib resistance. However, cardiovascular safety is an unfortunate but emerging challenge in CML and ALL patients treated with not only imatinib but also more so with the newer generations of BCR-ABL inhibitors. Among various TKI induced toxicities, vascular events including cardiac and cerebral ischemic events and peripheral arterial occlusive disease have become an emerging type of toxicity in CML patients. Damage by these drugs is largely due to inhibition of ABL (on-target effects). For example, imatinib-induced CTX has been shown to be mediated by a mutant of ABL, to which imatinib does not bind completely, rescues imatinib mediated toxicity. CML patients, that acquire imatinib resistance due to presence of T315I mutation and show sensitivity to Ponatinib, may manifest cardiac and vascular toxicity due to ABL1 inhibition. Accordingly, the degree of ABL1 and ABL2 repression along with the duration of repression will likely determine the level of CTX observed in these patients. ABL (ABL1) and Arg (ABL2) are a family of non-receptor tyrosine kinases characterized by unique carboxy-terminal, actin-binding domains that can bundle actin. The ABL kinases regulate a variety of cytoskeletal processes including membrane ruffling, chemotaxis and cell spreading. ABL-dependent regulation of the Rho-ROCK-myosin signalling pathway is critical for the maintenance of adherens junctions. Inhibition of the ABL kinases (either by Imatinib or by knockout or knockdown) results in the activation of RhoA and its downstream target ROCK, leading to enhanced phosphorylation of the myosin regulatory light chain (MLC) (FIG. 18). These signalling events result in enhanced stress fiber formation and increased actomyosin contractility, thereby disrupting adherens junctions. Altogether, data suggests that loss of ABL1 or ABL2 (Arg) or their inhibition results in reduced p190Rho GAP phosphorylation resulting in increased RhoA activity and activity of its downstream substrate ROCK (FIG. 18). Active ROCK mediates the downstream effects of RhoA on actin cytoskeleton.

Active ROCK mediates the downstream effects of RhoA on actin cytoskeleton. Based on these results, highly potent TKIs, which provide significant anti-CML/ALL benefit against 1st and 2^{nd} generation TKI resistant/relapsed CML/ALL patients, will likely inhibit endogenous ABL kinase activation in cardiomyocytes, VSMCs and ECs (FIG. 18). Given that inhibition of ABL in RhoA dependent manner results in hyperactivation of ROCK, leading to disruption of cytoskeletal functions including apoptosis, genetic loss of ROCK1 systemically and in a tissue specific manner will likely reverse and/or mitigate TKI induced CTX in mouse models of CML/ALL.

To investigate the involvement of ROCK kinases in CTX and vascular injury due to TKIs in the context of CML (naïve WT BCR-ABL, resistant T315I BCR-ABL and ALL-p190BCR-ABL) or ALL, a well-established and most widely used mouse bone marrow cell transduction-transplantation model will be utilized. Reports have shown that bone marrow cells transduced with the empty vector (MIEG3) and transplanted remain free of hematologic disorders within the 6-month observation period. By contrast, approximately 3 to 4 weeks after transplantation 100% mice bearing BCR-ABL(+) or BCR-ABL^{T315I}(+) cells develop CML, including splenomegaly, neutrophilia and leukocytosis. Mice will be evaluated weekly with a peripheral blood count using automated cell counter. Mice with leukocytosis of WBC>20,000/μl and <40,000/μl are considered diseased. These mice will be treated with TKIs including imatinib, dasatinib, nilotinib and ponatinib starting at one week post-transplantation. At least two different drug doses will be used for a period of 3 weeks as described (see e.g., Peng, C. & Li, S. CML mouse model in translational research. METHODS MOL BIOL 602, 253-266 (2010)). For example, imatinib (100 and 200 mg/kg/day), dasatinib (10 and 30 mg/kg/day) and ponatinib (20 and 30 mg/kg/day) will be used.

CML and ALL disease will be propagated in 6 distinct recipient mice as described in Example 7. Recipient mice will be of the following 6 genotypes: 1) WT; 2) ROCK1^{-/-} (systemic ablation); 3) ROCK1^{fl/fl}-MHC-Cre (cardiac specific ablation of ROCK1); 4) ROCK1^{fl/fl}-SM22α (vascular smooth muscle cell specific ablation of ROCK1); 5) ROCK1^{fl/fl}-Tie2-Cre (vascular endothelial cell specific ablation of ROCK1) and 6) ROCK1-MHC Tg mice (overexpression of ROCK1 in cardiomyocytes). These mice will be lethally irradiated and transplanted with bone marrow cells bearing BCR-ABL p210 or BCR-ABL-T315I or p190BCR-ABL as described (see e.g., Peng, C. & Li, S. CML mouse model in translational research. METHODS MOL BIOL 602, 253-266 (2010)). After 7 days, drug treatment will be initiated for 3 weeks with various drugs as described above. the ability of these drugs to reduce disease burden and CTX as described above in Example 7 will be assessed. Drug treated mice and vehicle treated mice will be monitored for peripheral blood counts and EGFP expression for an additional 4 weeks on a weekly basis. After 4 weeks of monitoring peripheral blood counts, the mice will be sacrificed and histological examination will be performed to determine the signs of splenomegaly, myeloid cell infiltration and/or EGFP expression in the spleen and BM. A cohort of mice will be followed for survival after stopping the drug treatment. At the time of sacrifice, the mice will also be examined for CTX and vascular injury as described above.

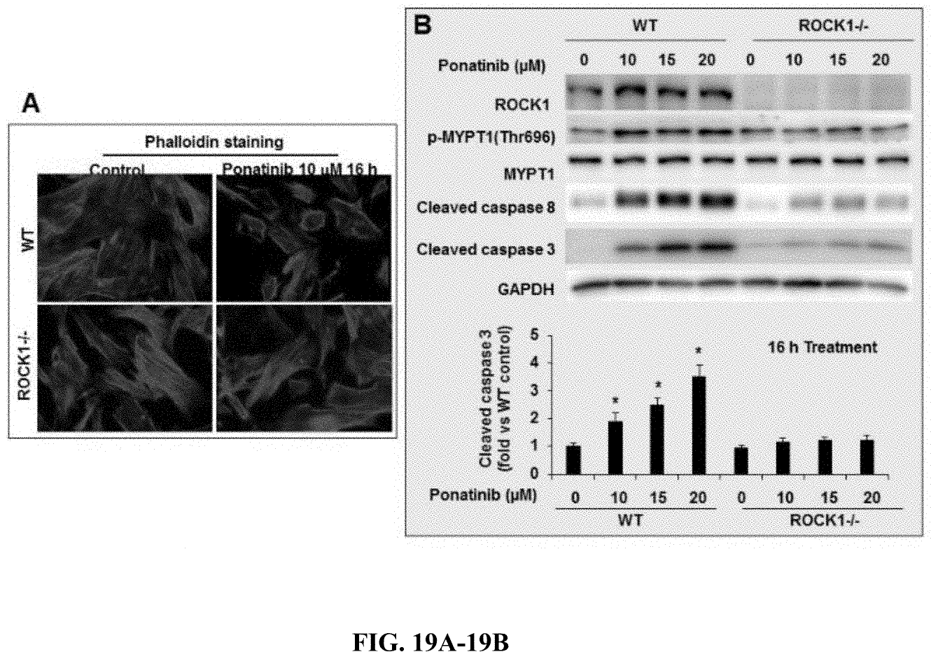

In addition, to further assess vascular injury, mesenteric arterioles, carotid artery and thoracic aorta will be examined in the background of ROCK1 deficiency. To measure ROCK activity in these tissues, the phosphorylation of MYPT1 on Thr696 and 853 and phosphorylation of MLC on Ser19 and Thr18/Ser19 will be examined by Western blot analysis as the phosphorylation on these sites reflect non-muscle myosin II activation by ROCK. To evaluate vascular remodeling, Hematoxylin/eosin staining of transverse sections will be performed to quantitate wall thickness and cross-sectional area, picrosirius red staining to quantitate collagen fibers, and immunostaining with anti-SM α-actin to quantitate neointima formation 75, respectively. In vivo, vascular leakage will be measured by Evans Blue assay, which measures blood vessel permeability. Mice will be treated IP for 3 days with TKIs once a day. On day 3, 30 minutes after the administration of drugs, Evans blue will be injected through tail vein. Mice will be euthanized after 30 minutes, mesenteric arterioles, carotid artery and thoracic aorta will be collected into solvent and Evans blue concentration will be measured. Given that TKI, such as dasatinib significantly increases the amount of Evans Blue in the small intestine, loss of ROCK1 will likely be protective. The above described functional changes will be correlated with changes in the status of ABL1 and ABL2 phosphorylation and these changes will be evaluated with the activation of ROCK and MLC. In preliminary studies, fibroblasts from WT and ROCK1^{-/-} mice have been isolated and treated with increasing doses of ponatinib. Referring now to FIG. 19, significant changes in ponatinib induced cell shape were noted in WT fibroblasts, which was not the case in ROCK1^{-/-} fibroblasts. Further, an increase in ROCK1 expression was observed in WT fibroblasts treated with increasing concentration of ponatinib, which was associated with increased activation of ROCK1 as assessed by MYPT1 phosphorylation. This increase in ROCK activation led to a profound increase in cleaved caspase 8/3 levels. Moreover, a profound decrease in cleaved caspase 3/8 levels was seen in ROCK1^{-/-} cells, which was associated with enhanced survival.

### Statistical Considerations

The basic experiment in this Example is to compare the CTX between the control and various ROCK1 lacking and overexpressing mice treated with TKIs among six groups of recipients. The data will be analyzed using a one-way ANOVA followed by t-test with the multiple comparison adjusted by Tukey's method. A sample size of 20 mice per group will provide 81.2% power to detect a large effect of 1.2 with a 5% type 1 error.

CTX and vascular injury will likely be mitigated in mice lacking ROCK1 and transplanted with CML cells when exposed to various TKIs. This will be associated with reduced activation of MLC and ROCK in mice treated with TKIs. The CTX will likely be most strongly reversed in CML mice treated with ponatinib in the absence of ROCK1. Also, a rescue in the vascular injury will be observed in mice lacking ROCK1 in VSMCs, ECs and cardiomyocytes but not to the same extent as seen in mice completely lacking ROCK1. These changes in human ECs, VSMCs and cardiomyocytes upon TKI treatment will further be assessed. While the hyperactivation of ROCK in response to TKIs is in part due to ABL inhibition, the activation of PDGR, VEGFR, and additional receptors that might be targeted as a result of “off-target” effects of some of these TKIs will be examined.

### Example 9: Use of Statins, Nitric Oxide (NO), and ROCK Inhibitor During the Treatment of CML with TKIs

The use of statins, nitric oxide (NO) and ROCK inhibitor during the treatment of CML with TKIs will likely mitigate CTX while maintaining high anti-leukemic activity. This will be examined in a model of CML.

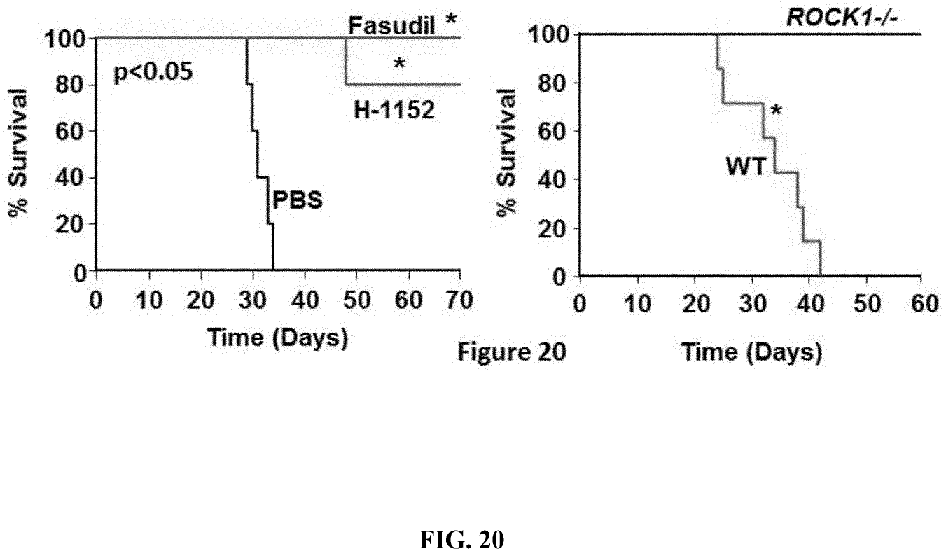

Increased ROCK activity is associated with cardiovascular disease (CVD). Administration of exogenous NO in patients with CVD inhibits ROCK activity and improves CV function. Given that TKIs induce ROCK activation and CTX in CML patients, exogenous NO will likely inhibit ROCK activity and show protective CV effects in CML mice treated with ponatinib. In humans, atorvastatin (statin) inhibits ROCK activity in patients with atherosclerosis and improves CVD. Administration of statins in CML bearing mice treated with ponatinib will likely rescue CTX. Finally, administration of a ROCK inhibitor, Fasudil, which has potent anti-leukemic activity in vivo models of AML (as potent as AML cells devoid of ROCK1), will show significant cardioprotective effects in CML mice treated with ponatinib. FIG. 20 provides significant enhancement in survival of the leukemic mice treated with Fasudil or H-1152, or the leukemic cells lacking ROCK1 compared to vehicle treated or WT leukemic cells.

BCR-ABL (both WT and T315I) expressing bone marrow donor cells will be prepared as described in Example 8. These cells will be transplanted into C57/BL6-CD45.1 recipient mice as described above. A week after transplantation, recipient mice will be randomly distributed into the following groups: 1) vehicle; 2) ponatinib (25 mg/kg/day); 3) NO (45 mg/kg/day, H-1026, a NO-donating nonsteroidal agent) plus ponatinib; 4) statin [pravastatin](10 mg/kg/day) plus ponatinib; and 5) Fasudil (50 mg/kg/day) plus ponatinib. All of these drugs have been extensively used in humans. The CML mice will be treated one week post-transplant for 4 weeks with either NO, statin or Fasudil. The following week (second week post-transplant), the mice will be treated with ponatinib for next 3 weeks. At the end of the drug treatment, a cohort of mice will be sacrificed for detailed anti-leukemic and CTX analysis and some will be followed long-term for survival and subsequent analysis as described in significant detail in above Examples. Co-administration of these drugs along with ponatinib will likely demonstrate both anti-leukemic effects as well as cardiac protection and mitigate vascular injury normally seen with ponatinib. In contrast, mice that are not treated with Fasudil, NO, or statin will likely show severe CTX, abnormal cardiac functions and vascular injury.

### Statistical Considerations

The sample size for the mouse experiment is calculated to detect large biological meaningful difference. The basic experiment in this Example is to compare the CTX between the control and various drug treated mice among five groups of recipients. The data will be analyzed using a one-way ANOVA followed by t-test with the multiple comparison adjusted by Tukey's method. A sample size of 20 mice per group will provide 81.2% power to detect a large effect of 1.2 with a 5% type 1 error.

Treatment of mice bearing CML with Fasudil, NO, statins in the presence of ponatinib will likely enhance the anti-leukemic effect of ponatinib and prolong the survival of leukemic mice but also protect these mice from CTX. This will likely be due to the inhibition of ROCK activity by ponatinib-induced ABL repression.

While the novel technology has been illustrated and described in detail in the figures and foregoing description, the same is to be considered as illustrative and not restrictive in character, it being understood that only the preferred embodiments have been shown and described and that all changes and modifications that come within the spirit of the novel technology are desired to be protected. As well, while the novel technology was illustrated using specific examples, theoretical arguments, accounts, and illustrations, these illustrations and the accompanying discussion should by no means be interpreted as limiting the technology. All patents, patent applications, and references to texts, scientific treatises, publications, and the like referenced in this application are incorporated herein by reference in their entirety.

1. A composition, comprising:
at least one anti-cancer agent; and
at least one agent that reduces the activity of at least one Rho/Rho Kinase pathway.

2. The composition according to claim 1, wherein the at least one anti-cancer agent comprises at least one anthracycline (ANT) and/or at least one tyrosine kinase inhibitor (TKI).

3. The composition according to claim 1, wherein the at least one anti-cancer agent comprises at least one anthracycline (ANT), and wherein the at least one anthracycline (ANT) comprises daunorubicin, doxorubicin, epirubicin, idarubicin, pixantrone, sabarubicin, and/or valrubicin, or a derivative thereof.

4. The composition according to claim 1, wherein the at least one anti-cancer agent comprises at least one tyrosine kinase inhibitor (TKI), and wherein the at least one tyrosine kinase inhibitor (TKI) comprises imatinib, dasatinib, nilotinib, bosutinib, ponatinib, lapatinib, adavosertib, gefitinib, erlotinib, toceranib, sorafenib, nilotinib, afatinib, axitinib, cabozantinib, osimertinib, lenvatinib, midostaurin, neratinib, regorafenib, and/or vandetanib, or a derivative thereof.

5. The composition according to claim 1 wherein the at least one agent that reduces the activity of the Rho/Rho kinase pathway comprises at least one Rho inhibitor and/or at least one Rho kinase (ROCK) inhibitor.

6. The composition according to claim 1, wherein the at least one agent that reduces the activity of at least one Rho/Rho Kinase pathway comprises at least one Rho inhibitor, and wherein the at least one Rho inhibitor comprises rhosin, Y16, and/or C3 Transferases, or a derivative thereof.

7. The composition according to claim 1, wherein the at least one agent that reduces the activity of at least one Rho/Rho Kinase pathway comprises at least one Rho kinase (ROCK) inhibitor, and wherein the at least one Rho kinase (ROCK) inhibitor comprises Fasudil, H-1152, Ripasudil, RKI-1447, Y-27632, GSK429286A, Y-30141, and/or nitric oxide (NO), or a derivative thereof.

8. The composition according to claim 1, further comprising at least one statin comprising atorvastatin, fluvastatin, lovastatin, pitavastatin, pravastatin, rosuvastatin, simvastatin, and/or ezetimibe/simvastatin combination, or a derivative thereof.

9. The composition according to claim 1, wherein the at least one anti-cancer agent and the at least one agent that reduces the activity of at least one Rho/Rho Kinase pathway are present in the composition in a ratio such that the composition exhibits synergy.

10. A method of treating a cancer, comprising the steps of:
administering to a subject diagnosed with a cancer a therapeutically effective dose of the composition according to any one of claim 1 or a pharmaceutically acceptable salt or metabolite thereof.

11. (canceled)

12. The method according to claim 10, wherein the cancer comprises bone cancer, brain cancer, breast cancer, endocrine cancer, gastrointestinal cancer, gynaecologic cancer, head and neck cancer, hematologic cancer, lung cancer, prostate cancer, renal cell carcinoma, skin cancer, urologic cancer, rare cancers, hematologic malignancies, lymphoma, leukemia, acute lymphoblastic leukemia (ALL), acute myeloid leukemia (AML), chronic lymphocytic leukemia (CLL), and/or chronic myeloid leukemia (CML).

13. The method according to claim 10, wherein the therapeutically effective dose of the composition of claim 1 comprises from 10 mg/kg/day to 1000 mg/kg/day, from 10 mg/kg/day to 500 mg/kg/day, from 10 mg/kg/day to 200 mg/kg/day, from 10 mg/kg/day to 100 mg/kg/day, from 10 mg/kg/day to 50 mg/kg/day, from 20 mg/kg/day to 1000 mg/kg/day, from 20 mg/kg/day to 500 mg/kg/day, from 20 mg/kg/day to 200 mg/kg/day, from 20 mg/kg/day to 100 mg/kg/day, or any combination thereof.

14. (canceled)

15. A method of reducing a side effect of a therapeutic regime, comprising the steps of:
administering to a subject at least one therapeutically effective dose of at least one agent that reduces the activity of at least one Rho/Rho kinase pathway;
wherein the subject has received at least one therapeutic regime comprising surgery, chemotherapy, radiation therapy, bone marrow transplant, targeted therapy, precision medicine, immunotherapy, stem cell transplant, hyperthermia, photodynamic therapy, blood product donation and transfusion, Light Amplification by Stimulated Emission of Radiation (LASER) in cancer treatment, DNA-damaging radiotherapy, endocrine therapy, and/or hormone therapy, and wherein the subject experiences at least one side effect.

16. The method according to claim 15, wherein the at least one therapeutically effective dose of at least one agent that reduces the activity of at least one Rho/Rho Kinase pathway comprises at least one Rho inhibitor and/or at least one Rho kinase (ROCK) inhibitor.

17. The method according to claim 15, wherein the at least one agent that reduces the activity of at least one Rho/Rho Kinase pathway comprises at least one Rho inhibitor, and wherein the at least one Rho inhibitor comprises rhosin, Y16, and/or C3 Transferases, or a derivative and/or a pharmaceutical salt thereof.

18. The method according to claim 15, wherein the at least one agent that reduces the activity of at least one Rho/Rho Kinase pathway comprises at least one Rho kinase (ROCK) inhibitor, and wherein the at least one Rho kinase (ROCK) inhibitor comprises Fasudil, H-1152, Ripasudil, RKI-1447, Y-27632, GSK429286A, Y-30141, and/or nitric oxide (NO), or a derivative and/or a pharmaceutical salt thereof.

19. The method according to claim 15, wherein the therapeutic regime comprises administration of at least one therapeutic agent comprising at least one anthracycline (ANT) and/or at least one tyrosine kinase inhibitor (TKI).

20. The method according to claim 19, wherein the at least one anthracycline (ANT) comprises daunorubicin, doxorubicin, epirubicin, idarubicin, pixantrone, sabarubicin, and/or valrubicin, or a derivative thereof.

21. The method according to claim 19, wherein the at least one tyrosine kinase inhibitor (TKI) comprises imatinib, dasatinib, nilotinib, bosutinib, ponatinib, lapatinib, adavosertib, gefitinib, erlotinib, toceranib, sorafenib, nilotinib, afatinib, axitinib, cabozantinib, osimertinib, lenvatinib, midostaurin, neratinib, regorafenib, and/or vandetanib, or a derivative thereof.

22. The method according claim 15, wherein the therapeutically effective dose of the at least one agent that reduces the activity of at least one Rho/Rho Kinase pathway comprises from 10 mg/kg/day to 1000 mg/kg/day, from 10 mg/kg/day to 500 mg/kg/day, from 10 mg/kg/day to 200 mg/kg/day, from 10 mg/kg/day to 100 mg/kg/day, from 10 mg/kg/day to 50 mg/kg/day, from 20 mg/kg/day to 1000 mg/kg/day, from 20 mg/kg/day to 500 mg/kg/day, from 20 mg/kg/day to 200 mg/kg/day, from 20 mg/kg/day to 100 mg/kg/day, or any combination thereof.

23. (canceled)

24. (canceled)
